(19) United States
(12) Patent Application Publication
(10) Pub. No.: US 2025/0339024 A1
(43) Pub. Date: Nov. 6, 2025

(54) OPHTHALMIC INFORMATION PROCESSING APPARATUS, OPHTHALMIC SYSTEM, OPHTHALMIC INFORMATION PROCESSING METHOD, AND RECORDING MEDIUM

(71) Applicant: TOPCON CORPORATION, Tokyo (JP)

(72) Inventors: Masahiro AKIBA, Wako-shi (JP); Makoto FUJINO, Tokyo (JP); Tsutomu KIKAWA, Tokyo (JP)

(73) Assignee: TOPCON CORPORATION, Tokyo (JP)

(21) Appl. No.: 19/273,173

(22) Filed: Jul. 18, 2025

Related U.S. Application Data
(63) Continuation of application No. PCT/JP2024/001048, filed on Jan. 17, 2024.
(60) Provisional application No. 63/441,328, filed on Jan. 26, 2023.

(51) Int. Cl. A61B 3/10; A61B 3/00; A61B 5/00; G06T 7/00; G16H 30/00; G16H 50/30

(52) U.S. Cl. CPC A61B 3/102; A61B 3/0025; A61B 5/7275; G06T 7/0012; G16H 30/00; G16H 50/30; G06T 2207/10101; G06T 2207/20056; G06T 2207/20081; G06T 2207/30041

(57) ABSTRACT

An ophthalmic information processing apparatus includes an acquisition unit and an information processor. The acquisition unit is configured to acquire one or more interferograms obtained by performing OCT scan on an eye of an examinee. The information processor is configured to execute generation processing of medical service supporting information that supports a provision of a medical service for the examinee, based on the one or more interferograms. The information processor is configured to execute at least a part of the generation processing using a learned model generated in advance by performing machine learning.

### CROSS-REFERENCE TO RELATED APPLICATIONS

The present application is a continuation application of International Patent Application No. PCT/JP2024/001048, filed Jan. 17, 2024, which claims priority to U.S. Provisional application, Ser. No. 63/441,328, filed Jan. 26, 2023, both of which are herein incorporated by reference in their entirety.

### FIELD

The disclosure relates to an ophthalmic information processing apparatus, an ophthalmic system, an ophthalmic information processing method, and a recording medium.

### BACKGROUND

Optical coherence tomography (OCT) apparatuses that are used to form images representing the surface morphology or the internal morphology of an object to be measured using light beam emitted from a laser light source or the like have been known. OCT performed in the OCT apparatuses is not invasive on the human body, and therefore is expected to be applied to the medical field or the biological field, in particular. For example, in the ophthalmic field, apparatuses for forming images of the fundus, the cornea, or the like have been in practical use. Such apparatuses using a method of OCT (OCT apparatuses) can be applied to observe tomographic structure of various sites of an eye. In addition, because of the ability to acquire high-definition images, the OCT apparatuses are applied to the diagnosis of various eye diseases.

Japanese Unexamined Patent Application Publication No. 2022-062160 discloses a method of acquiring interferograms of the fundus using OCT, and of acquiring OCT images by performing fast Fourier transformation, etc. on the acquired interferograms.

“Quantifying frequency content in cross-sectional retinal scans of diabetics vs. controls” (J. A. Papay, A. E. Elsner, PLOS ONE 16(6): e0253091, https://doi.org/10.1371/journal.pone.0253091, Jun. 18, 2021) discloses that the distribution of spatial frequencies obtained by performing fast Fourier transformation on the OCT image can be a diagnostic criterion for diabetic retinopathy.

### SUMMARY

One aspect of embodiments is an ophthalmic information processing apparatus including an acquisition unit and an information processor. The acquisition unit is configured to acquire one or more interferograms obtained by performing OCT scan on an eye of an examinee. The information processor is configured to execute generation processing of medical service supporting information that supports a provision of a medical service for the examinee, based on the one or more interferograms. The information processor is configured to execute at least a part of the generation processing using a learned model generated in advance by performing machine learning.

Another aspect of the embodiments is an ophthalmic system including an OCT optical system and the ophthalmic information processing apparatus described above. The OCT optical system is configured to perform OCT scan on an eye of an examinee. The ophthalmic information processing apparatus is configured to acquire one or more interferograms from the OCT optical system.

Still another aspect of the embodiments is an ophthalmic information processing method including an acquisition step and an information processing step. The acquisition step is performed to acquire one or more interferograms obtained by performing OCT scan on an eye of an examinee. The information processing step is performed to execute generation processing of medical service supporting information that supports a provision of a medical service for the examinee, based on the one or more interferograms. The information processing step is performed to execute at least a part of the generation processing using a learned model generated in advance by performing machine learning.

Still another aspect of the embodiments is a computer readable non-transitory recording medium in which a program for causing a computer to execute each step of the ophthalmic information processing method described above is recorded.

### BRIEF DESCRIPTION OF THE DRAWINGS

FIG. 1 is a schematic diagram illustrating an example of a configuration of an ophthalmic system according to embodiments.

FIG. 2 is a schematic diagram illustrating an example of a configuration of an OCT apparatus according to the embodiments.

FIG. 3 is a schematic diagram illustrating an example of a configuration of an ophthalmic information processing apparatus according to the embodiments.

FIG. 4 is a schematic diagram illustrating an example of a configuration of the ophthalmic information processing apparatus according to the embodiments.

FIG. 5 is a flowchart illustrating an example of an operation of the ophthalmic information processing apparatus according to the embodiments.

FIG. 6 is a flowchart illustrating an example of an operation of the ophthalmic information processing apparatus according to the embodiments.

FIG. 7 is an explanatory diagram of the operation of the ophthalmic system according to the embodiments.

FIG. 8 is a schematic diagram illustrating an example of a configuration of the ophthalmic information processing apparatus according to a first modification example of the embodiments.

FIG. 9 is a flowchart illustrating an example of an operation of the ophthalmic information processing apparatus according to the first modification example of the embodiments.

FIG. 10 is an explanatory diagram of an operation of the ophthalmic system according to the first modification example of the embodiments.

FIG. 11 is a flowchart illustrating an example of an operation of the ophthalmic information processing apparatus according to a second modification example of the embodiments.

FIG. 12 is an explanatory diagram of an operation of the ophthalmic system according to the second modification example of the embodiments.

FIG. 13 is a flowchart illustrating an example of an operation of the ophthalmic information processing apparatus according to a third modification example of the embodiments.

FIG. 14 is an explanatory diagram of an operation of the ophthalmic system according to the third modification example of the embodiments.

FIG. 15 is a flowchart illustrating an example of an operation of the ophthalmic information processing apparatus according to a fourth modification example of the embodiments.

FIG. 16 is an explanatory diagram of an operation of the ophthalmic system according to the fourth modification example of the embodiments.

FIG. 17 is a flowchart illustrating an example of an operation of the ophthalmic information processing apparatus according to a fifth modification example of the embodiments.

FIG. 18 is an explanatory diagram of an operation of the ophthalmic system according to the fifth modification example of the embodiments.

FIG. 19 is a schematic diagram illustrating an example of a configuration of the ophthalmic apparatus according to a sixth modification example of the embodiments.

### DETAILED DESCRIPTION

In general, in the arithmetic processing for acquiring OCT images from the interferograms as disclosed in Japanese Unexamined Patent Application Publication No. 2022-062160, predetermined processing is performed for the purpose of suppressing mirror images, etc. so that a site of interest can be observed in more detail. This means that loss of information amount of signal components will inevitably occur in the course of the arithmetic processing described above. When analysis processing such as acquiring diagnostic supporting information, acquiring supporting information for classifying disease types, improving images by reducing speckles and noise, identifying regions of interest, or identifying desired layer regions is performed on OCT images acquired in this manner, the accuracy and reliability of the analysis result further decrease.

On the other hand, in the method disclosed in “Quantifying frequency content in cross-sectional retinal scans of diabetics vs. controls” described above, redundant arithmetic processing is performed. As a result, the efficiency of use of arithmetic processing resource decreases, and the speed of the arithmetic processing also decreases.

According to some embodiments according to the present invention, a new technique for acquiring analysis result of an eye of an examinee with good accuracy and reliability can be provided. In addition, according to the present invention, a new technique for efficiently acquiring analysis result of the eye with high precision can be provided.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

Referring now to the drawings, exemplary embodiments of an ophthalmic information processing apparatus, an ophthalmic system, an ophthalmic information processing method, and a program (recording medium) according to the present invention are described below. Any of the contents of the documents cited in the present specification and arbitrary known techniques may be applied to the embodiments below.

An ophthalmic information processing apparatus according to embodiments is configured to acquire one or more interferograms obtained by performing OCT scan on an eye of an examinee (subject). The ophthalmic information processing apparatus is configured to execute generation processing of medical service supporting information that supports a provision of a medical service for the examinee, based on the acquired one or more interferograms.

Here, the OCT scan is a measurement for a spatial dimension in a one-dimensional axis direction, which means that the interference signals (interferograms) are acquired as signals for the one-dimensional wavenumber dimension. The OCT scan is part of OCT measurement or OCT imaging (photographing). The interferogram is information that represents the wavenumber dependence (wavelength dependence) of the interference intensity, interference spectrum information, interference spectrum signal, or A-scan data. The interferogram can be acquired using an OCT optical system. The OCT optical system includes an interference optical system. The interference optical system is configured to split light from a light source into measurement light and reference light, to irradiate the measurement light onto the eye of the examinee, and to detect interference light between returning light of the measurement light from the eye and the reference light that has passed through a reference arm. The OCT optical system can output detection result(s) of the interference light as the interferogram(s).

In case that two or more interferograms are acquired using the OCT optical system, the two or more interferograms are acquired by entering the measurement light into two or more incident positions (scan positions), which are different from each other, on the eye. In some embodiments, the two or more interferograms are acquired by sequentially entering the measurement light into each of the two or more incident positions, which are different from each other, on the eye at timings different from each other. For example, the two or more incident positions are arranged along a B-scan direction, which intersects the A-scan direction, with reference to a predetermined incident position. For example, the two or more incident positions are positioned separately from each other within a single region of interest. For example, the two or more incident positions are positioned in respective two or more regions of interest that are separated from each other.

Alternatively, the two or more interferograms may be acquired by entering the measurement light into approximately the same incident position on the eye at timings different from each other. In this case, changes in the time series of the acquired two or more interferograms enable the detection of minute red blood cells (erythrocytes) flowing in blood vessels as motion contrast.

In some embodiments, the ophthalmic information processing apparatus is configured to acquire the one or more interferograms from an OCT optical system provided outside the ophthalmic information processing apparatus. In some embodiments, the ophthalmic information processing apparatus is configured to acquire the one or more interferograms from an OCT apparatus or a server apparatus, which is connected via a network. Here, the OCT apparatus is provided with the OCT optical system. In some embodiments, the ophthalmic information processing apparatus is configured to acquire the one or more interferograms from the OCT optical system housed in the same housing as the ophthalmic information processing apparatus.

Further, the medical service supporting information is information that supports the provision of medical services to the examinee. The medical service supporting information may be provided using at least one of images, numbers, texts (character strings), sounds, light, or vibrations. For example, the medical service supporting information include an analysis result of the morphology of the tomographic structure of the eye of the examinee, OCT images of the eye of the examinee (broadly speaking, reflectance intensity distribution), or information suggesting the possibility of disease in the eye of the examinee (such as numerical values of the examination). Examples of the analysis result of the morphology of the tomographic structure of the eye of the examinee includes an analysis result (OCT analysis result) that could not be obtained conventionally without analyzing the OCT image. Examples of the information suggesting the possibility of diseases in the eye of the examinee include information suggesting the possibility of the eye that could not be obtained conventionally without analyzing OCT image. In embodiments related to the present invention, the ophthalmic information processing apparatus is configured to generate medical service supporting information directly from the interferogram(s) using a learned model described below, regardless of the presence or absence of OCT images. Examples of the OCT image include an A-scan image, a B-scan image, a C-scan image, a projection image, an en-face image, a shadowgram, an OCT angiography (OCTA) image, a three-dimensional OCT image, and a tomographic image representing the morphology of the tomographic structures in a desired cross-sectional direction. The medical service supporting information may include the content of the provided medical service, the frequency of the provided medical service, the type of the provided medical service, the grade of the provided medical service, the time when the medical service should be provided, or information on institutions providing the medical service.

The ophthalmic information processing apparatus according to the embodiments is configured to execute at least a part of generation processing of the medical service supporting information described above using a learned model, which is generated in advance by performing machine learning, to output the medical service supporting information. Here, the ophthalmic information processing apparatus is preferably configured to perform processing equivalent to at least the Fourier transformation processing (fast Fourier transformation processing) among the generation processing of the medical service supporting information described above, using the learned model.

The learned model may be any configuration that can be trained using machine learning. The learned model is generated in advance so as to output the medical service supporting information by performing machine learning using at least the one or more interferograms as input. The learned model may further be configured to input at least one of an image, a number, a text, or sound information.

Thereby, the medical service supporting information is directly generated from the one or more interferograms using the learned model obtained by performing machine learning. Therefore, for the interferograms, complex signal processing for forming OCT images is no longer necessary. As a result, the loss of information amount accompanying signal processing can be suppressed, and the analysis result of the eye of the examinee can be obtained with good accuracy and precision, or with high precision and efficiency.

In addition, the ophthalmic information processing apparatus may use the learned model solely for the part of the processing equivalent to a part of the generation processing of the medical service supporting information to generate the medical service supporting information. Even in this case, compared to the case where all signal processing is performed, the loss of information amount accompanying signal processing can be suppressed, and the analysis result of the eye of the examinee can be obtained with good accuracy and precision, or with high precision and efficiency.

The ophthalmic system according to the embodiments includes the OCT optical system configured to acquire the one or more interferograms of the eye of the examinee using OCT, and the ophthalmic information processing apparatus according to the embodiments. In this case, the ophthalmic information processing apparatus is configured to acquire the one or more interferograms from the OCT optical system.

One aspect of the ophthalmic system is a system including an OCT apparatus with the OCT optical system, and the ophthalmic information processing apparatus according to the embodiments. In this case, the OCT apparatus and the ophthalmic information processing apparatus are configured to be capable of being connected via a wired or wireless communication path (network). The ophthalmic information processing apparatus is configured to acquire the one or more interferograms from the OCT optical system via the communication path.

Another aspect of the ophthalmic system is a system including an OCT apparatus with the OCT optical system, a server apparatus (cloud server apparatus), and the ophthalmic information processing apparatus according to the embodiments. In this case, the OCT apparatus and the server apparatus are configured to be capable of being connected via a wired or wireless communication path. The server apparatus and the ophthalmic information processing apparatus are configured to be capable of being connected via a wired or wireless communication path. The server apparatus is configured to store the one or more interferograms obtained by the OCT optical system in association with the examinee. The ophthalmic information processing apparatus is configured to acquire the one or more interferograms stored in the server apparatus, via the communication path.

Still another aspect of the ophthalmic system is an ophthalmic apparatus in which the OCT optical system and the ophthalmic information processing apparatus according to the embodiments are housed in the same housing.

An ophthalmic information processing method according to the embodiments includes one or more steps for realizing the processing executed by a processor (computer) in the ophthalmic information processing apparatus according to the embodiments. A program according to the embodiments causes the processor to execute each step of the ophthalmic information processing method according to the embodiments. In other words, the program according to the embodiments is a computer program comprising instructions which, when the program is executed by a computer, cause the computer to carry out the ophthalmic information processing method according to the embodiments. A recording medium (storage medium) according to the embodiments is any computer readable non-transitory recording medium (storage medium) on which the program according to the embodiments is recorded.

The recording medium may be an electronic medium using magnetism, light, magneto-optical, semiconductor, or the like. Typically, the recording medium is a magnetic tape, a magnetic disk, an optical disk, a magneto-optical disk, a flash memory, a solid state drive, or the like. Further, the program may be transmitted and received through a network such as the Internet, LAN, etc.

The term “processor” as used herein refers to a circuit such as, for example, a central processing unit (CPU), a graphics processing unit (GPU), an application specific integrated circuit (ASIC), and a programmable logic device (PLD). Examples of PLD include a simple programmable logic device (SPLD), a complex programmable logic device (CPLD), and a field programmable gate array (FPGA). The processor realizes, for example, the function according to the embodiments by reading out a computer program stored in a storage circuit or a storage device and executing the computer program.

Hereinafter, the configurations according to the embodiments will be described specifically.

Hereinafter, an image acquired by using OCT may sometimes be referred to as “OCT image”. Further, the measurement operation for forming OCT images may be referred to as OCT measurement. Furthermore, the “image data” and an “image” based on the image data may not be distinguished from each other in the present specification.

### [Ophthalmic System]

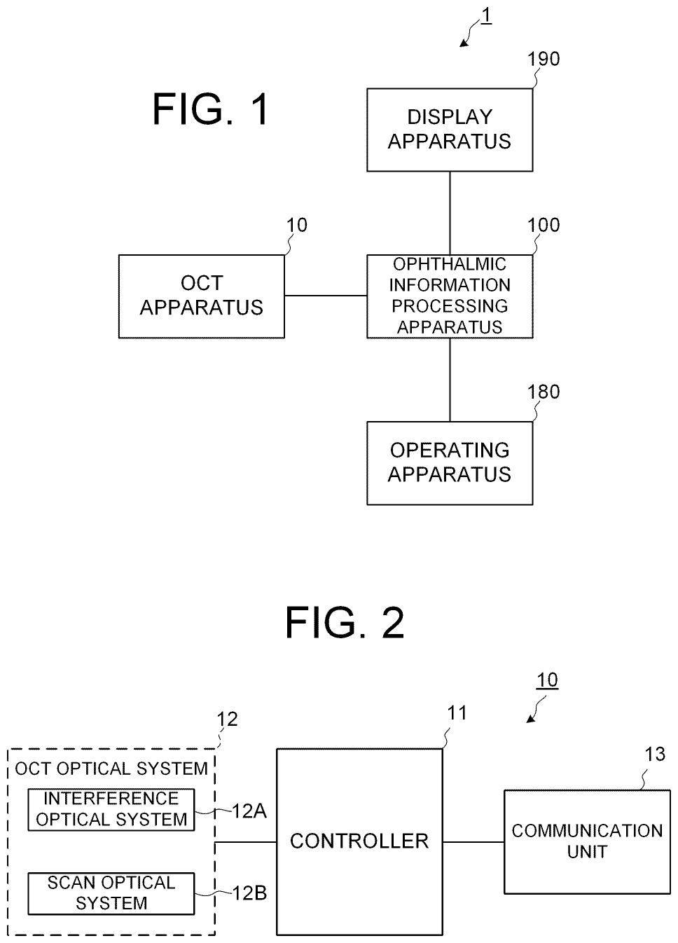

FIG. 1 shows a block diagram of an example of a configuration of the ophthalmic system according to the embodiments. The ophthalmic system 1 according to the embodiments includes an OCT apparatus 10, an ophthalmic information processing apparatus 100, an operating apparatus 180, and a display apparatus 190. It should be noted that the configuration of the ophthalmic system 1 shown in FIG. 1 is merely one aspect of the embodiments. The configuration of the ophthalmic system 1 according to the embodiments may be a configuration in which the OCT apparatus 10 and the ophthalmic information processing apparatus 100 are integrated, or a configuration in which the OCT apparatus 10, the ophthalmic information processing apparatus 100, the operating apparatus 180, and the display apparatus 190 are integrated, as described above.

The OCT apparatus 10 includes an OCT optical system, and is configured to acquire the one or more interferograms by performing OCT scan on the eye of the examinee. The OCT apparatus 10 is configured to send data including the acquired one or more interferograms to the ophthalmic information processing apparatus 100.

In some embodiments, the OCT apparatus 10 and the ophthalmic information processing apparatus 100 are connected via a data communication network (LAN, WAN, or peer-to-peer network). The ophthalmic information processing apparatus 100 according to some embodiments receives data including one or more interferograms from one of a plurality of ophthalmic apparatuses 10 selectively connected via the data communication network.

The ophthalmic information processing apparatus 100 acquires the one or more interferograms from the OCT apparatus 10, and executes generation processing of the medical service supporting information that supports a provision of a medical service for the examinee, based on the acquired one or more interferograms. The ophthalmic information processing apparatus 100 executes at least a part of the generation processing of the medical service supporting information using a learned model generated in advance by performing machine learning on a learning model.

The operating apparatus 180 and the display apparatus 190 provide the function for exchanging information between the ophthalmic information processing apparatus 100 and the user, such as displaying information, inputting information, and inputting operation instructions, as a user interface unit. The operating apparatus 180 includes an operating device such as a lever, a button, a key, and pointing device. The operating apparatus 180 according to some embodiments includes a microphone for inputting information using sound. The display apparatus 190 includes a display device such as a flat-panel display. In some embodiments, the functions of the operating apparatus 180 and the display apparatus 190 are realized using a device in which a device having an input function such as a touch panel display and a device having a display function are integrated. In some embodiments, the operating apparatus 180 and the display apparatus 190 include a graphical user interface (GUI) for inputting and outputting information.

### [OCT Apparatus]

FIG. 2 shows a block diagram of an example of a configuration of the OCT apparatus 10 according to the embodiments.

The OCT apparatus 10 includes an optical system for acquiring the one or more interferograms of the eye of the examinee. For example, the OCT apparatus 10 is provided with an optical system of a swept source OCT. However, the type of OCT provided with the OCT apparatus 10 is not limited to swept source OCT, and it may be a spectral domain OCT or the like. The swept source OCT is a method of splitting light from a wavelength swept type (i.e., a wavelength scanning type) light source into measurement light and reference light, of making returning light of the measurement light having traveled through the eye of the examinee as an object to be measured and the reference light having traveled through the reference optical path interfere with each other to generate interference light, and of detecting the interference light. The spectral domain OCT is a method of splitting light from a low-coherence light source into the measurement light and the reference light, of making returning light of the measurement light having traveled through the examinee as an object to be measured and the reference light having traveled through a reference optical path interfere with each other to generate interference light, and of detecting the interference light. The photographing site (measurement site) of the eye of the examinee is, for example, a fundus or an anterior segment.

The OCT apparatus 10 includes a controller 11, an OCT optical system 12, and a communication unit 13.

The controller 11 controls each part of the OCT apparatus 10. In particular, the controller 11 controls the OCT optical system 12 and the communication unit 13.

The OCT optical system 12 acquires one or more interferograms of the eye of the examinee, by performing OCT scan on the eye of the examinee using OCT. The OCT optical system 12 includes an interference optical system 12A and a scan optical system 12B.

The interference optical system 12A splits light from a light source (wavelength swept type light source) into measurement light and reference light, irradiates the measurement light onto the eye of the examinee, makes returning light of the measurement light having traveled through the eye of the examinee and the reference light having traveled through a reference optical path interfere with each other to generate interference light, and detects the interference light. The interference optical system 12A includes at least a first fiber coupler, a second fiber coupler, or an optical receiver such as a balanced photodiode. The first fiber coupler is configured to split the light from the light source into the measurement light and the reference light. The second fiber coupler is configured to make the returning light of the measurement light having traveled through the eye of the examinee interfere with the reference light having passed through the reference optical path to generate the interference light. For example, the optical receiver is configured to receive the interference light generated by the second fiber coupler in synchronization with the wavelength swept timings from the light source. The interference optical system 12A may include the light source.

The scan optical system 12B changes an incident position of the measurement light on the photographing site (for example, fundus or anterior segment) of the eye of the examinee by deflecting the measurement light generated by the interference optical system 12A, under the control of the controller 11. The scan optical system 12B includes, for example, an optical scanner disposed at a position substantially conjugate optically to a pupil of the eye of the examinee. The optical scanner includes, for example, a first galvanometer mirror that deflects the measurement light in a horizontal direction, a second galvanometer mirror that deflects the measurement light in a vertical direction, and a mechanism that independently drives the first galvanometer mirror and the second galvanometer mirror. For example, the second galvanometer mirror is configured to further deflect the measurement light deflected by the first galvanometer mirror. Thereby, the measurement light can be deflected in arbitrary directions on a scan plane.

A detection result (detection signal) of the interference light obtained by the interference optical system 12A is signal(s) (interference signal(s)) indicating the spectrum of the interference light as the interferogram(s). In a state where the deflection direction by the scan optical system 12B is fixed, one interferogram can be acquired by entering the measurement light into one incident position on the eye (photographing site). By sequentially entering the measurement light into two or more incident positions on the eye (photographing site) while changing the deflection direction using the scan optical system 12B, the two or more interferograms can be sequentially acquired. Alternatively, by sequentially entering the measurement light into a single incident point at timings different from each other on the eye (photographing site), the two or more interferograms can be sequentially acquired.

The controller 11 includes a control processor. The control processor realizes, for example, the function according to the embodiments by reading out a computer program stored in a storage circuit or a storage device and executing the computer program. At least a part of the storage circuit or the storage apparatus may be included in the processor. Further, at least a part of the storage circuit or the storage apparatus may be provided outside of the processor.

The storage apparatus, etc. stores various types of data. Examples of the data stored in the storage apparatus, etc. include the one or more interferograms acquired by the OCT optical system 12, information related to the examinee, and information related to the eye of the examinee. The storage apparatus, etc. may store a variety of computer programs and data for operating each part of the OCT apparatus 10.

The communication unit 13 performs communication interface processing for transmitting or receiving information with the ophthalmic information processing apparatus 100 under the control of the controller 11.

The OCT apparatus 10 according to some embodiments includes a fundus camera for acquiring an image of the fundus of the eye of the examinee, an anterior segment camera for acquiring an image of the anterior segment of the eye of the examinee, a scanning laser ophthalmoscope, or a slit lamp microscope. In some embodiments, the fundus image acquired by the fundus camera is a fluorescein fluorescence fundus angiogram or a fundus autofluorescnece inspection image.

### [Ophthalmic Information Processing Apparatus]

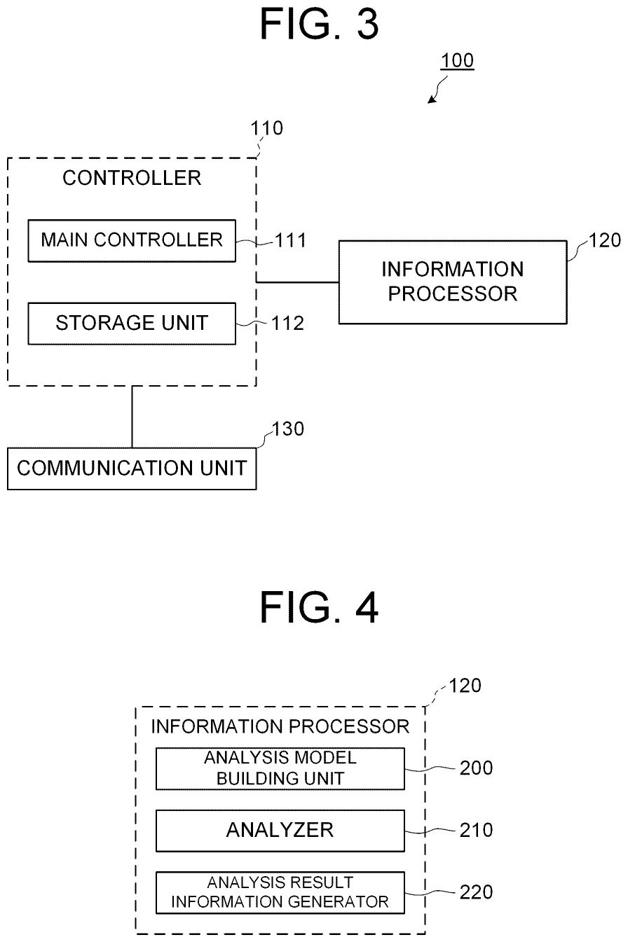

FIG. 3 and FIG. 4 show block diagrams of examples of the configuration of the ophthalmic information processing apparatus 100 according to the embodiments. FIG. 4 shows a block diagram of an example of the configuration of an information processor 120 in FIG. 3.

The ophthalmic information processing apparatus 100 includes a controller 110, the information processor 120, and a communication unit 130.

The controller 110 controls each part of the ophthalmic information processing apparatus 100. In particular, the controller 110 controls the information processor 120 and the communication unit 130. The controller 110 includes a main controller 111 and a storage unit 112.

The controller 110 can control each part of the ophthalmic system 1 based on operation instruction signal corresponding to the operation content of the user on the operating apparatus 180.

Each of the controller 110 and the information processor 120 includes one or more processors. The functions of the information processor 120 are realized by one or more information processing processors. In some embodiments, both of the functions of the controller 110 and the information processor 120 are realized by a single processor.

The storage unit 112 stores various types of data. Examples of the data stored in the storage unit 112 include the one or more interferograms acquired from the OCT apparatus 10, information processing result acquired by the information processor 120, information related to the examinee, and information related to the eye of the examinee. The storage unit 112 may store a variety of computer programs and data for the operation of each part of the ophthalmic information processing apparatus 100.

The communication unit 130 performs communication interface processing for transmitting or receiving information with the communication unit 13 in the OCT apparatus 10 under the control of the controller 110.

The information processor 120 is configured to execute generation processing of medical service supporting information that supports a provision of a medical service for the examinee, based on the one or more interferograms acquired from the OCT apparatus 10.

The information processor 120 executes the generation processing of the medical service supporting information using an analysis model generated in advance by performing machine learning on a learning model. The analysis model is a learned model that uses at least the one or more interferograms as input data and the medical service supporting information as output data. The analysis model is generated in advance so as to output the medical service supporting information by performing machine learning using the one or more interferograms as input.

Examples of the medical service according to the embodiments include diagnosis, examination (detailed examination), judgment, and interpretation (image reading). The medical service supporting information according to the embodiments is information that supports the appropriate provision of the medical service described above for the examinee. The content of the medical service supporting information according to the embodiments may include information corresponding to the type of the medical service.

### (First Example of Medical Service Supporting Information)

The first example of the medical service supporting information is supporting information including at least one of OCT analysis result(s) for supporting the diagnosis of the examinee by doctors, or the like who are provided with the information, or an OCT image of the eye of the examinee. Examples of the OCT analysis result according to the present example include supporting information that supports a determination of presence or absence of a disease, supporting information that supports a determination of presence of absence of a risk of developing a disease, supporting information that supports a determination of a type of a disease, supporting information that supports a determination of a severity of a disease, supporting information that supports a decision of a treatment of a disease, or supporting information that supports a prediction of a disease symptom (condition, state). The supporting information described above may include statistical information that can be identified through statistical processing. Examples of the OCT image according to the present example include an OCT image of a photographing region (scan region) from which the interferograms are acquired, and an OCT image in which a site related to a disease (site of lesion, site of interest) is identifiably depicted.

Examples of the information that supports a determination of presence or absence of a disease include information indicating the presence or absence of the disease, information indicating the possibility of the presence or absence of the disease, and information indicating a confidence of the presence or absence of the disease. Examples of the information that supports a determination of presence of absence of a risk of developing a disease include information indicating the presence or absence of the risk of developing the disease, information indicating a degree of the risk of developing the disease, information indicating a risk factor that influences developing the disease, information indicating a position of the examinee (eye) within a distribution of patients who developed the disease, and information indicating a confidence of the presence or absence of the risk of developing the disease. The information that supports a determination of presence of absence of a risk of developing a disease may be presented for each disease (type), for each predetermined risk factor of developing the disease, or for each site that influences the increase or decrease of the risk of developing the disease or the accuracy of the risk of developing the disease. Examples of the supporting information that supports a determination of a type of a disease include information indicating the type of the disease, information indicating the possibility of the presence or absence of the disease for each type, and information indicating a confidence of the presence or absence of the disease for each type. Examples of the supporting information that supports a determination of a severity of a disease include information indicating the severity of the disease, information indicating the possibility of the presence or absence of the disease for each severity, and information indicating a confidence of the presence or absence of the disease for each severity. Examples of the supporting information that supports a decision of a treatment of a disease include information indicating a treatment of the disease, information indicating whether or not the treatment should be selected for each treatment, and information indicating a confidence for each treatment. Examples of the supporting information that supports a prediction of a disease symptom include information indicating a symptom that is currently present or may appear in the future, information indicating the possibility of symptom for each symptom that is currently present or may appear in the future, and information indicating a confidence of symptom for each symptom that is currently present or may appear in the future.

Examples of the disease include an eye disease and a systemic disease (illness). Examples of the eye disease include an anterior eye disease and a posterior eye disease. Examples of the anterior eye disease include cataract, dry eye, pterygium, keratitis, and corneal endothelial damage. Examples of the posterior eye disease include glaucoma, age-related macular degeneration, myopia; hypermetropia (hyperopia); astigmatism (high myopia), posterior vitreous detachment (PVD), optic neuropathy, central serous chorioretinopathy (CSC), diabetic retinopathy, uveitis, Behcet disease, pigmentary retinal degeneration, retinal vein occlusion, retinal detachment, retinal break, and retinal hole. Examples of the systemic disease include hypertension, arterial sclerosis, diabetes, Alzheimer's disease, atopic dermatitis, graves disease, and blood disease.

Examples of the type of the disease include type identified by classifying the disease, such as cataract, dry eye, glaucoma, age-related macular degeneration, diabetic retinopathy, uveitis, pigmentary retinal degeneration, retinal vein occlusion, hypertension, arterial sclerosis, Alzheimer's disease, and blood disease, using a general method.

Examples of the type of the cataract include a type (nuclear cataract, etc.) that can be identified by classifying according to an opacified site of a crystalline lens, a type (incipient cataract, etc.) that can be identified by classifying according to degree of progression, and a type (senile cataract, etc.) that can be identified by classifying according to a cause of disease. Examples of the type of the dry eye include a type (evaporative dry eye, etc.) that can be identified by classifying according to a cause of disease. Examples of the type of the glaucoma include a type (primary glaucoma, etc.) that can be identified by classifying according to corner angle findings, a disease, and a cause and a type (Nicolela's classification) that can be identified by classifying according to a shape of an optic disc.

Examples of the type of the age-related macular degeneration include a type (soft drusen, etc.) that can be identified by classifying according to a precursor lesion and a type (exudative age-related macular degeneration, etc.) that can be identified by classifying according to a geographic atrophy, a choroidal neovascularization, and the like. Examples of the type of the diabetic retinopathy include a type (simple retinopathy, etc.) that can be identified by classifying according to a disease stage and a type (proliferative retinopathy, etc.) that can be identified by classifying according to a degree of severity. Examples of the type of the uveitis include a type (anterior uveitis, etc.) that can be identified by classifying according to an affected site. Examples of the type of the pigmentary retinal degeneration include a type (typical retinitis pigmentosa, etc.) that can be identified by classifying according to a site of disturbance and a symptom and a type that can be identified by classifying according to a degree of severity. Examples of the type of the retinal vein occlusion include a type (branch retinal vein occulusion (BRVO), etc.) that can be identified by classifying according to an occlusion site. Examples of the type of the hypertension include a type (Scheie's classification, etc.) that can be identified by classifying according to a diameter of the retinal artery and a hemorrhage. Examples of a type of the arterial sclerosis include a type (Keith-Wagener classification, etc.) that can be identified by classifying according to a vascular narrowing, a hemorrhage, a leukoma, and an edema. Examples of the type of the Alzheimer's disease include a type (vascular dementia, etc.) that can be identified by classifying according to a blood flow.

Examples of the treatment of the disease include surgical remedy (cataract surgery, refractive surgery, vitreous surgery), medical therapy (anti-VEGF (vascular endothelial growth factor) medical remedy), photodynamic therapy (PDT), laser treatment (laser photocoagulation), and an intraocular lens.

Further, as described above, the medical service supporting information may include an OCT image in which at least one of the regions of interest or the lesion sites of the disease described above is identifiably depicted.

Furthermore, the medical service supporting information may include the analysis result of the layer region such as the retina. The analysis result of the layer region is represented using at least one of the image, the number, or the text. For example, the medical service supporting information includes an OCT image with the analysis result(s) of the layer regions superimposed, or a map representing a distribution information of the analysis result(s) of the layer region(s).

Examples of the layer region of the retina include the inner limiting membrane, the nerve fiber layer, the ganglion cell layer, the inner plexiform layer, the inner nuclear layer, the outer plexiform layer, the outer nuclear layer, the external limiting membrane, the photoreceptor layer, the retinal pigment epithelium layer (RPE), and the choroid. In addition, the layer region of the retina may include the photoreceptor inner/outersegment junction (IS/OS) or the ellipsoid zone (EZ), and the chorio-scleral interface (CSI). In some embodiments, the layer region corresponding to the layer tissue such as a Bruch membrane, a choroid, a sclera or a vitreous body is identified. For example, the layer region corresponding to the layer tissue with a predetermined number of pixels on the sclera side with respect to the RPE is defined as the Bruch membrane. Examples of the analysis result of the layer region include a result of the layer region segmentation, a layer region thickness map, and layer region thickness distribution information (thickness map).

Furthermore, the medical service supporting information may include a higher quality OCT image with improved image quality, such as speckle reduction and noise reduction.

### (Second Example of Medical Service Supporting Information)

The second example of the medical service supporting information is supporting information including at least one of OCT analysis result(s) for supporting the examination of the examinee by doctors, examiners, or the examinee who are provided with the information, or an OCT image of the eye of the examinee. Examples of the OCT analysis result according to the present example include supporting information that supports a determination of at least one of necessity of an examination of the eye of the examinee, a degree of urgency of the examination of the eye of the examinee, the frequency of the examination of the eye of the examinee, the site to be examined in the examination of the eye of the examinee, the content (type) of the examination of the eye of the examinee, or the examination institute (medical institution, etc.) of the examination of the eye of the examinee. The supporting information described above may include statistical information that can be identified through statistical processing. Examples of the OCT image according to the present example include an OCT image of a photographing region from which the interferograms are acquired, and an OCT image in which a site related to the examination target is identifiably depicted.

The medical service supporting information according to the second example may further include at least one of the information included in the medical service supporting information according to the first example.

### (Third Example of Medical Service Supporting Information)

The third example of the medical service supporting information is supporting information including at least one of OCT analysis result(s) for supporting the determination of the examination result(s) of the eye of the examinee by doctors, examiners, or the examinee who are provided with the information, or an OCT image of the eye of the examinee. Examples of the OCT analysis result according to the present example include supporting information that supports a determination of at least one of a quality of an examination result of the eye of the examinee, a severity of the examination result of the eye of the examinee, or an examination result to be noted of the eye of the examinee. The supporting information described above may include statistical information that can be identified through statistical processing. Examples of the OCT image according to the present example include an OCT image of a photographing region from which the interferograms are acquired, and an OCT image in which a site that may affect the examination result(s) is identifiably depicted.

The medical service supporting information according to the third example may further include at least one of the information included in the medical service supporting information according to the first example and the second example.

### (Fourth Example of Medical Service Supporting Information)

The fourth example of the medical service supporting information is supporting information including at least one of OCT analysis result(s) for supporting the interpretation (image reading) of the examination image of the eye of the examinee by doctors, examiners, or the examinee who are provided with the information, or an OCT image of the eye of the examinee. Examples of the OCT analysis result according to the present example include supporting information that supports at least one of more rapid and easier interpretation, a determination of the site to be noted, or a determination of the interpretation result. The supporting information described above may include statistical information that can be identified through statistical processing. Examples of the OCT image according to the present example include an OCT image of a photographing region from which the interferograms are acquired, an OCT image with added information to facilitate interpretation, and an OCT image in which a site to be noted or a site that may affect the interpretation result(s) is identifiably depicted.

The medical service supporting information according to the fourth example may further include at least one of the information included in the medical service supporting information according to the first example, the second example, and the third example.

Further, the medical service supporting information according to the embodiments may include information for additionally training the learned model. For example, the medical service supporting information may include supporting information that supports an annotation, such as labeling and marking.

Furthermore, the medical service supporting information according to the embodiments may include adjustment parameter(s) for adjusting optical condition(s) of the OCT optical system 12.

As shown in FIG. 4, the information processor 120 includes an analysis model building unit (analysis model builder) 200, an analyzer 210 and an analysis result information generator 220. In some embodiments, the analysis model building unit 200 is provided outside (e.g., an external server apparatus) of the ophthalmic information processing apparatus 100. In this case, the ophthalmic information processing apparatus 100 is configured to acquire the analysis model from outside the apparatus, and to execute the generation processing of the medical service supporting information describe above using the acquired analysis model.

In some embodiments, of the functions of the information processor 120 are realized by a single information processor. In some embodiments, the processor that realizes the functions of the analysis model building unit 200 is provided separately from the information processing processor that realizes the functions of the analyzer 210 and the analysis result information generator 220.

The analysis model building unit 200 builds an analysis model as a learned model, by performing machine learning on a learning model that uses one or more interferograms as input and outputs the medical service supporting information described above. The data set that defines a configuration of the analysis model built by the analysis model building unit 200 is stored in the storage unit 112 (or an external server apparatus not shown).

The analyzer 210 acquires the one or more interferograms received from the OCT apparatus 10 via the communication unit 130. The analyzer 210 generates the medical service supporting information from the acquired one or more interferograms, using the analysis model previously built by the analysis model building unit 200.

The analysis result information generator 220 generates the analysis result information including the medical service supporting information, which is the analysis result(s) obtained by the analyzer 210. The controller 110 (main controller 111) can cause the analysis result information generated by the analysis result information generator 220 to be displayed on a display means, such as the display apparatus 190.

### (Analysis Model Building Unit 200)

The analysis model according to the embodiments may be a learned model having any configuration that can be trained using machine learning. The analysis model may be a single learned model or may be configured to include two or more learned models.

For example, information other than two-dimensional distribution information and three-dimensional distribution information among the medical service supporting information can be obtained using an analysis model with a convolutional neural network (CNN) structure. Here, examples of the two-dimensional distribution information or the three-dimensional distribution information include an image such as the OCT image, a two-dimensional thickness map, and a three-dimensional OCT image.

The CNN includes a convolution layer, a downsampling layer (pooling layer), a fully-connected layer, and an output layer. The CNN may include a plurality of units, each of which includes the convolution layer and the downsampling layer. The plurality of units is configured by connecting each unit in a plurality of states. In each unit, for example, inputs of the downsampling layers are connected to outputs of the convolution layers.

The convolution layer performs convolution processing on the input data using kernels according to the kernel size, kernel value, stride value, padding value, and dilation value. The downsampling layer performs downsampling processing on the input data. For example, the downsampling layer performs downsampling processing on data input from the preceding convolution layers. Examples of the downsampling processing include max pooling processing and average pooling processing. The fully-connected layer combines data generated via the one or more convolutional layers and the one or more downsampling layers into a single node, and generates feature variables (feature map) transformed by a predetermined activation function. The output layer generates confidence from the feature variables input from all fully-connected layers using an activation function such as a softmax function or an identity function.

In the CNN, weighing factors (coefficients) as adjustable parameters (learning parameters) are assigned between neurons in the two layers connected to each other. The weighting factors assigned between the neurons and the kernel values of the convolution layers are updated by machine learning. In some embodiments, at least one of the kernel size, the stride value, the padding value, or the dilation value is updated by machine learning. Each neuron performs calculation using a response function on calculation result in which weighting factor(s) from the one or more input neurons is/are added, and outputs the obtained calculation result to a neuron in the next stage.

The CNN having the configuration as described above repeats the extraction of the feature map (feature) and the downsampling (filtering) for each predetermined region with respect to the one or more input interferograms to extract the feature map of the one or more interferograms. The CNN outputs the medical service supporting information according to machine learning performed in advance, based on the extracted feature map(s).

Examples of the CNN include VGG16, VGG19, InceptionV3, ResNet18, ResNet50, Xception, and DenseNet201.

For example, the two-dimensional distribution information and the three-dimensional distribution information among the medical service supporting information can be obtained using an analysis model with a fully convolution network (FCN) configuration.

The FCN includes a convolution layer, a downsampling layer, a deconvolution layer, an up-sampling layer, and an output layer. The FCN may include a plurality of units, each of which includes the convolution layer and the downsampling layer. The plurality of units is configured by connecting each unit in a plurality of states. In each unit, for example, inputs of the downsampling layers are connected to outputs of the convolution layers. In addition, the FCN may include a plurality of units, each of which includes the convolution layer and the up-sampling layer. The plurality of units is configured by connecting each unit in a plurality of states. In each unit, for example, inputs of the up-sampling layers are connected to outputs of the convolution layers.

The up-sampling layer performs up-sampling processing on the input data. For example, the up-sampling layer performs the up-sampling processing on the feature extracted by the convolution layers and the downsampling layers or the data input from the preceding deconvolution layers. Examples of the up-sampling processing include duplication and interpolation. The deconvolution layer performs deconvolution processing on the input data using kernels according to the kernel size, the kernel value, the stride value, the padding value, and the dilation value.

Also in the FCN, weighing factors (coefficients) as adjustable parameters (learning parameters) are assigned between neurons in the two layers connected to each other. The weighting factors assigned between the neurons, the kernel values of the convolution layers, and the kernel values of the deconvolution layers are updated by machine learning. In some embodiments, at least one of the kernel size, the stride value, the padding value, or the dilation value of at least one of the convolution layer or the deconvolution layer is updated by machine learning. Each neuron performs calculation using a response function on calculation result in which weighting coefficient(s) from one or more input neurons is/are added, and outputs the obtained calculation result to a neuron in the next stage.

The FCN having the configuration as described above repeats the extraction of the feature map (feature) and the downsampling for each predetermined region with respect to the one or more input interferograms using the convolution layer(s) and the downsampling layer(s). Thereby, the final feature map of the one or more interferograms is extracted. The FCN repeats the expansion of the feature map and the up-sampling using the deconvolution layer(s) and the up-sampling layer(s) on the final feature map extracted. Thereby, while retaining the positional information of one or more input interferograms, the medical service supporting information indicating the two-dimensional distribution information reflecting feature maps is output in accordance with machine learning performed in advance.

Examples of the FCN include U-net.

The three-dimensional distribution information among the medical service supporting information can be obtained using a network (neural network) with a configuration that is an extension of the FCN configuration described above.

The analysis model building unit 200 can learn a learning model by machine learning using a known method such as gradient descent method and error back-propagation method. For example, the analysis model building unit 200 builds the analysis model (learned model) by learning the learning model having the configuration as described above using training data, by known machine learning such as supervised learning, unsupervised learning, or semi-supervised learning. The training data includes at least one or more interferograms as input data.

In the case of supervised learning, the analysis model building unit 200 builds the analysis model by performing machine learning of the learning model using the training data with ground truth labels as teaching data. The teaching data is ground truth data (correct answer data) that has been labeled with ground truth label(s) (or annotation(s)) in advance by doctors, or the like.

The teaching data may be a pair of output data and input data corresponding to the output data. Here, the output data is acquired using OCT of the same type as the type of OCT (type of OCT of the OCT optical system 12) from which the interferograms have been acquired. In other words, in case that the OCT optical system 12 is configured to acquire interferograms using swept source OCT, the teaching data is also a pair of output data acquired using swept source OCT and input data corresponding to the output data. Alternatively, in case that the OCT optical system 12 is configured to acquire interferograms using spectral domain OCT, the teaching data is also a pair of output data acquired using spectral domain OCT and input data corresponding to the output data. In some embodiments, the analysis model is a learned model configured to output the medical service supporting information by performing machine learning using the one or more interferograms and the type of OCT as input data.

When input data is input into the learning model, the output data is obtained according to the configuration of the learning model, which can be adjusted by parameters. Here, the parameter includes at least one of the weighing factor described above, the kernel size of the convolution layer or the deconvolution layer, the kernel value of the convolution layer or the deconvolution layer, the stride value of the convolution layer or the deconvolution layer, the padding value of the convolution layer or the deconvolution layer, or the dilation value of the convolution layer or the deconvolution layer. When the one or more teaching data are sequentially input to the learning model, the analysis model building unit 200 adjusts the parameters so that the output data are approximately matched to the ground truth data for all of the input teaching data. Thereby, the analysis model with a configuration whose parameters have been adjusted by supervised learning is built.

In the case of unsupervised learning, the analysis model building unit 200 builds the analysis model by performing machine learning of the learning model using one or more training data without ground truth labels. When the one or more training data are sequentially input to the learning model, the analysis model building unit 200 adjusts the parameters so that the input one or more training data are characterized. Thereby, the analysis model with a configuration whose parameters have been adjusted is built without generating teaching data.

In addition, the analysis model building unit 200 may build the analysis model by performing unsupervised learning on the learning model using a generative adversarial network (GAN) or a deep convolutional generative adversarial network (DCGAN). The GAN or the DCGAN includes a generator and a discriminator. The generator generates fake data from random noise. The discriminator determines whether real data matches the fake data. By adversarially repeating the learning of the generator so that the generator can generate fake data closer to real data and the learning of the discriminator so that the discriminator can discriminate between the real data and the fake data generated by the generator, the generator becomes capable of generating fake data that is close to real data. The configuration of the learned generator corresponds to the configuration of the analysis model according to the embodiments.

In the case of semi-supervised learning, the analysis model building unit 200 builds the analysis model by performing machine learning of the learning model using the teaching data and the training data without ground truth labels. For example, the analysis model building unit 200 builds the analysis model by performing adjustment of parameters by the unsupervised learning described above on the learned model that has been obtained by learning a learning model by the supervised learning described above. Alternatively, for example, the analysis model building unit 200 builds the analysis model by performing adjustment of parameters by the supervised learning described above on the learned model that has been obtained by learning a learning model by the unsupervised learning described above.

In addition, the analysis model building unit 200 may also build the analysis models by known machine learning such as reinforcement learning. Furthermore, the analysis model building unit 200 can update the parameters by performing additional machine learning on the analysis model described above to rebuild the configuration of the analysis model.

The input data for the analysis model may include one or more incidental data (supplementary data) in addition to the one or more interferograms. In this case, the analysis model building unit 200 builds the analysis model so as to perform machine learning described above using the one or more interferograms and the one or more incidental data as input so as to output the medical service supporting information described above.

Examples of the one or more incidental data include acquisition information of the one or more interferograms as the input data, the one or more interferograms or the OCT analysis result previously acquired for the same eye of the examinee, and background data indicating the characteristics and properties of the examinee related medical care. The one or more incidental data may include an OCT image formed based on the one or more interferograms as input data, or information indicating a layer region to be divided into desired regions. Further, the one or more incidental data may include information indicating the acquisition time interval of the interferograms for acquiring motion contrast data such as OCTA images. Examples of the acquisition information of the one or more interferograms include information indicating at least one of date and time of the OCT scan, the light quantity for photographing (light quantity of measurement light), photographing region (scan region), scan mode, the type of OCT, or the type of OCT apparatus (device model). Further, the acquisition information of the one or more interferograms may include at least one of the scan positions (the incident position of the measurement light) of each interferogram, or the site of the eye including the scan position(s).

Furthermore, the one or more incidental data may include information representing a recipient to which the medical service supporting information is provided. Examples of the recipient of the medical service supporting information include a doctor, an examiner, an examinee (subject), and an administrator (support manager) of the ophthalmic system. This allows the medical service supporting information to be appropriately provided according to the recipients.

For example, the analysis model building unit 200 can build the analysis model that can output OCTA image(s) by performing machine learning on the learning model using the teaching data. In this case, the teaching data may be data of a plurality of pairs of input data and output data. Here, the input data are interferograms at different acquisition times from each other and information indicating acquisition time intervals. The output data are OCTA images corresponding to the input data.

For example, the analysis model building unit 200 can build the analysis model that can output OCT image(s) with higher quality by performing machine learning on the learning model using the teaching data. In this case, the teaching data may be data of a plurality of pairs of input data and output data. Here, the input data are the one or more interferograms. The output data are OCT images with high quality according to the input data. Examples of the OCT image with high quality include OCT images include an image obtained by performing position matching a plurality of OCT images acquired by a plurality of OCT scans for the approximately same photographing region and performing averaging processing (additive averaging processing) on the OCT images on which the position matching has been performed, and an OCT image on which a predetermined high quality processing has been performed. This allows higher quality OCT images with speckle noise reduction and noise reduction to be obtained.

For example, the analysis model building unit 200 can build the analysis model that can output OCT image(s) in which segmentation processing (region division processing) has been performed on a desired layer region, by performing machine learning on the learning model using the teaching data. In this case, the teaching data may be data of a plurality of pairs of input data and output data. Here, the input data are the one or more interferograms. The output data are OCT images in which a desired layer region has been regionally divided according to the input data. In some embodiments, the input data is incidental to information representing the desired layer region. In this case, the teaching data may be data of a plurality of pairs of input data and output data. Here, the input data are the one or more interferograms and the information representing the desired layer region. The output data are OCT images in which a desired layer region has been regionally divided according to the input data.

For example, the analysis model building unit 200 can build the analysis model that can output OCT image(s) from which a desired layer region has been extracted, by performing machine learning on the learning model using the teaching data. In this case, the teaching data may be data of a plurality of pairs of input data and output data. Here, the input data are the one or more interferograms. The output data are OCT images from which a desired layer region has been extracted according to the input data. In some embodiments, the input data is incidental to information representing the desired layer region. In this case, the teaching data may be data of a plurality of pairs of input data and output data. Here, the input data are the one or more interferograms and the information representing the desired layer region. The output data are OCT images from which a desired layer region has been extracted according to the input data.

For example, the analysis model building unit 200 can build the analysis model that can output OCT image(s) in which a region of interest (or attention region) has been depicted in an identifiable manner, by performing machine learning on the learning model using the teaching data. In this case, the teaching data may be data of a plurality of pairs of input data and output data. Here, the input data are the one or more interferograms. The output data are OCT images in which a region of interest (or attention region) has been depicted in an identifiable manner according to the input data.

For example, the analysis model building unit 200 can build the analysis model that can output statistical OCT analysis result(s) by performing machine learning on the learning model using the teaching data. Examples of the statistical OCT analysis result include information indicating changes in OCT analysis results over time and information indicating regression analysis results of the time-series OCT analysis results. In this case, the teaching data may be data of a plurality of pairs of input data and output data. Here, the input data are interferograms at different acquisition times from each other and information indicating acquisition time intervals. The output data are statistical OCT analysis results according to the input data.

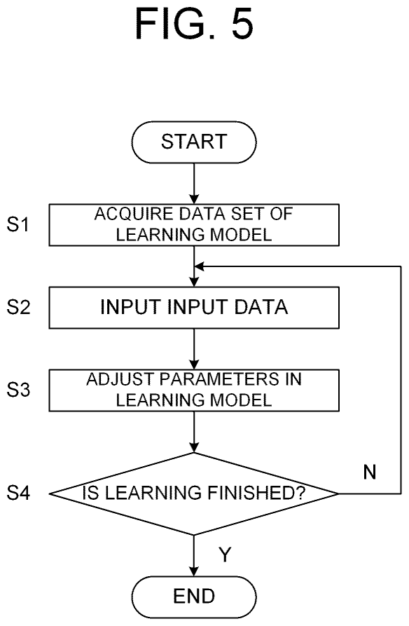

FIG. 5 shows a flowchart of an example of an operation of the analysis model building unit 200 in FIG. 4. FIG. 5 represents a flowchart of an example of an operation in case that the analysis model building unit 200 builds the analysis model by supervised learning. The storage unit 112 or a storage unit (not shown) in the information processor 120 stores a computer program for realizing the processing shown in FIG. 5. A processor configured to realize the functions of the analysis model building unit 200 operates according to the computer program, and thereby the processor performs the processing shown in FIG. 5.

In FIG. 5, it is assumed that the storage unit 112 or the storage unit (not shown) in the information processor 120 has stored initial values of the data set of the learning model targeted for machine learning in advance. In addition, it is assumed that the storage unit 112 or the storage unit (not shown) in the information processor 120 has stored groups of teaching data using supervised learning, in advance.

### (S1: Acquire Data Set of Learning Model)

First, the analysis model building unit 200 reads out data set of the learning model from the storage unit 112 or the storage unit (not shown) in the information processor 120.

### (S2: Input Input Data)

Next, the analysis model building unit 200 reads out groups of teaching data from the storage unit 112 or the storage unit (not shown) in the information processor 120. The analysis model building unit 200 inputs (enters) input data of one of the groups of teaching data read out into the learning model.

### (S3: Adjust Parameters in Learning Model)

Subsequently, the analysis model building unit 200 acquires the output data of the learning model from which the input data was input in step S2, and adjusts the parameters of the learning model using a known method such as error back-propagation method.

### (S4: Is Learning Finished?)

Subsequently, the analysis model building unit 200 determines whether or not the learning should be finished. For example, the analysis model building unit 200 determines whether or not the learning should be finished, by determining whether or not the parameters of the learning model have been adjusted using all of the predetermined groups of teaching data. For example, the analysis model building unit 200 determines whether or not the learning should be finished, by determining whether or not the differences between the output data of the learning model whose parameters have been adjusted and the teaching data have converged to within a predetermined threshold or less.

When it is determined that the learning should not be finished (S4: N), the processing of the analysis model building unit 200 proceeds to step S2.

When it is determined that the learning should be finished (S4: Y), the analysis model building unit 200 stores the learned model obtained by repeating step S3 as the analysis model in the storage unit 112 or in the storage unit (not shown) in the information processor 120. After that, the analysis model building processing is finished (END).

### (Analyzer 210)

The analyzer 210 generates the medical service supporting information based on the one or more interferograms from the OCT apparatus 10, using the analysis model described above. Specifically, the information processing processor that realizes the functions of the analyzer 210 reads out data set that defines the configuration of the analysis model from the storage unit 112 or the storage unit (not shown) in the information processor 120. The information processing processor executes the arithmetic processing based on the one or more interferograms to operate so as to output the medical service supporting information, according to instructions from the data set that has been read out.

In some embodiments, the analyzer 210 (or information processor 120) is configured to perform a predetermined preprocessing on the one or more interferograms and to output the medical service supporting information using the one or more interferograms after preprocessing as the input data. Examples of the preprocessing include adjustment of the size of the one or more interferograms, and position matching of the one or more interferograms. Examples of the adjustment of the size include adjustment to a size in a default depth direction of each interferogram and adjustment to match the sizes of the two or more interferograms. Examples of the position matching include aligning a position in the depth direction of each interferogram to a predetermined reference position, and aligning positions in the depth direction position of the two or more interferograms with reference to a scan center position of the measurement light or a characteristic site of the eye.

### (Analysis Result Information Generator 220)

The analysis result information generator 220 generates the analysis result information using the medical service supporting information obtained by the analyzer 210. The analysis result information is represented using at least one of the image, the number, the text, the sound, the light, or the vibration.

Examples of the analysis result information include information from which a part of the medical service supporting information is extracted, information in which the display format of all of the medical service supporting information is changed, and information obtained by synthesizing the medical service supporting information and information generated by the ophthalmic information processing apparatus 100 or an external device. Examples of synthesizing two sets of information include superimposing one set of information onto another, adding one set of information to another, associating recognizably correspondence relationship between one set of information and another, and comparably parallelizing information from one set of information from another.

The OCT optical system 12 or the communication unit 130 is an example of the “acquisition unit” according to the embodiments. The OCT image output by the medical service supporting information generation processing performed by analyzer 210 is an example of the “first OCT image” according to the embodiments. The OCT analysis result output by the medical service supporting information generation processing executed by analyzer 210 is an example of the “analysis result of a morphology of a tomographic structure of the eye” according to the embodiments. The controller 110 (main controller 111) is an example of the “display controller” according to the embodiments. The display apparatus 190 is an example of the “display means” according to the embodiments.

### [Operation Example]

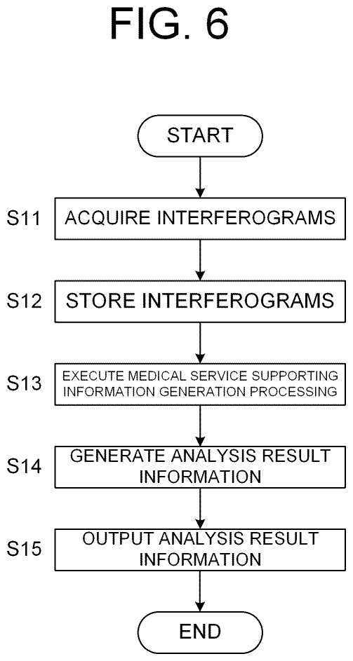

FIG. 6 shows an example of an operation of the ophthalmic information processing apparatus 100. The storage unit 112 or a storage unit (not shown) in the information processor 120 stores a computer program for realizing the processing shown in FIG. 6. One or more processors configured to realize the functions of the ophthalmic information processing apparatus 100 operates according to the computer program, and thereby the one or more processors perform the processing shown in FIG. 6.

In FIG. 6, it is assumed that the data set of the analysis model built by the analysis model building unit 200 is expanded in advance in the working memory of the information processor 120, the data set being read out from the storage unit 112 or the storage unit (not shown) in the information processor 120.

### (S11: Acquire Interferograms)

First, the main controller 111 controls the communication unit 130 to establish the communication connection between the communication unit 130 and the communication unit 13 in the OCT apparatus 10. The communication unit 130 performs interface processing with the communication unit 13 according to a predetermined communication protocol to establish the communication connection with the communication unit 13.

For example, prior to step S11, the OCT apparatus 10 has already acquired the one or more interferograms by performing OCT scan on the eye of the examinee using the OCT optical system 12. Alternatively, for example, the OCT apparatus 10 performs OCT scan on a predetermined photographing region (scan region) of the eye of the examinee using the OCT optical system 12, in response to instructions from the ophthalmic information processing apparatus 100 based on operation signals corresponding to the operation contents entered to the operating apparatus 180. The OCT apparatus 10 stores the one or more interferograms obtained by performing OCT scan.

The main controller 111 acquires the one or more interferograms stored in the OCT apparatus 10 via the communication path established with the communication unit 13 in the OCT apparatus 10.

### (S12: Store Interferograms)

Subsequently, the main controller 111 stores the one or more interferograms acquired in step S11 in the storage unit 112.

### (S13: Execute Medical Service Supporting Information Generation Processing)

Subsequently, the main controller 111 controls the analyzer 210 in the information processor 120 to execute the medical service supporting information generation processing using the analysis model with the one or more interferograms stored in step S12 as input data.

The analyzer 210 outputs the medical service supporting information described above as the output data, by inputting the one or more interferograms to the data set of the analysis model expanded in advance in the working memory.

### (S14: Generate Analysis Result Information)

Next, the main controller 111 controls the analysis result information generator 220 in the information processor 120 to generate the analysis result information of the eye of the examinee using the medical service supporting information obtained in step S13.

The analysis result information generator 220 generates the analysis result information including at least a part of the medical service supporting information, as described above.

### (S15: Output Analysis Result Information)

Next, the main controller 111 outputs the analysis result information generated in step S14. For example, the main controller 111 controls the display apparatus 190 to display the analysis result information generated in step S14, as a display controller. For example, the main controller 111 controls a printing device not shown to print the analysis result information generated in step S14, as an output controller. In some embodiments, the main controller 111 stores the analysis result information generated in step S24 in an external server device (not shown) in association with an identification number of the examinee.

This terminates the processing of the ophthalmic information processing apparatus 100 (END).

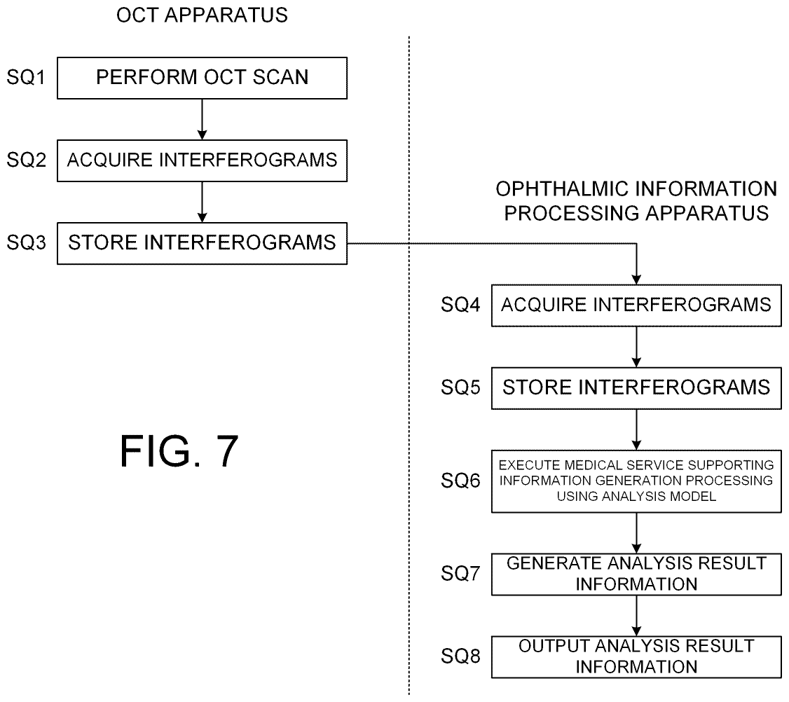

FIG. 7 shows an example of an operation sequence of the ophthalmic system 1 according to the embodiments. In FIG. 7, it is assumed that the analysis model has already been built by the analysis model building unit 200 and the data set of the analysis model has been stored in the storage unit 112 or the storage unit (not shown) in the information processor 120.

In the OCT apparatus 10, the controller 11 controls the OCT optical system 12 to perform OCT scan on the eye of the examinee under photographing conditions (photographing light quantity, photographing region) set in advance (SQ1).

The controller 11 acquires the one or more interferograms by sequentially acquiring the detection results of the interference light acquired by the OCT scan (SQ2), and stores the acquired one or more interferograms in the storage unit (not shown) in the controller 11 (SQ3).

In the ophthalmic information processing apparatus 100, the controller 110 controls the communication unit 130 to establish the communication connection with the OCT apparatus 10 to acquire the one or more interferograms from the OCT apparatus 10 (SQ4). After the one or more interferograms have been acquired by the OCT apparatus 10, the controller 11 may control the communication unit 13 to establish the communication connection with the ophthalmic information processing apparatus 100 and to transmit the one or more interferograms to the ophthalmic information processing apparatus 100.

The controller 110 stores the one or more interferograms acquired from the OCT apparatus 10 in the storage unit 112 or the storage unit in the information processor 120 (SQ5). Subsequently, the controller 110 reads out the one or more interferograms, and controls the analyzer 210 to execute the medical service supporting information generation processing using the one or more interferograms as input data (SQ6). The data set of the analysis model has been read out from the storage unit 112 or the storage unit (not shown) in the information processor 120, and has already been expanded in the working memory. The analyzer 210 outputs the medical service supporting information described above as the output data, by inputting the one or more interferograms to the data set of the analysis model expanded in the working memory.

The controller 110 controls the analysis result information generator 220 to generate the analysis result information using at least a part of the medical service supporting information output from the analysis model (SQ7). After that, the controller 110 controls the display apparatus 190, etc. to output the generated analysis result information (SQ8).

As explained above, in the embodiments, the medical service supporting information is generated directly from the one or more interferograms acquired by performing OCT scan on the eye of the examinee, using the analysis model obtained by machine learning. This eliminates the need for complex signal processing on the acquired interferograms.

Conventionally, a reflection intensity distribution (reflection intensity profile) is calculated by performing predetermined signal processing on the acquired interferograms, and an OCT image is formed by imaging the reflection intensity distribution and arranging it in one-dimensional direction, two-dimensional directions, or three-dimensional directions. This signal processing includes noise reduction processing, rescaling processing, apodization processing, variance compensation processing, fast Fourier transformation processing, logarithmic transformation processing, luminance transformation processing, and histogram adjustment processing, etc. These processing depend on the configuration of the OCT optical system for acquiring the interferograms and/or the optical conditions for acquiring the interferograms, resulting in highly complex processing. This required advanced software technology and increased calculation processing time, leading to higher computation costs. When extended to three-dimensional data, the calculation processing time becomes even longer, leading to a further increase in calculation costs.

In addition, the signal processing described above inevitably results in some loss of information, even when advanced processing parameters are adjusted.

For example, in the noise reduction processing, it is very difficult to completely remove alone the noise component based on unwanted light, which may remove part of the signal component.

For example, in the rescaling processing, some of the signal components may be removed during the conversion from wavelength components to wavenumber components.

For example, in the apodization processing, depending on the processing parameters (such as window size) of the apodization function and/or accuracy of the apodization function, the accuracy of the processing results may decrease, or some signal components may be removed as the amplitude of the side lobes decreases. In addition, adjustment of processing parameters and apodization functions becomes complicated.

For example, in the variance compensation processing, depending on the processing parameters (such as coefficients) of the variance compensation function and/or accuracy of the variance compensation function, the accuracy of the processing results may decrease. In addition, adjustment of processing parameters and variance compensation functions becomes complicated.

For example, in the fast Fourier transformation processing, theoretically, truncation errors and discretization errors may cause a decrease in the accuracy of the amplitude components of signal components.

For example, in the logarithmic transformation processing and the luminance transformation processing, some of the signal components may be removed when the amplitude components of the signal components are logarithmically converted and luminance conversion is performed. In addition, adjustment of processing parameters in these processing becomes complicated.

For example, in the histogram adjustment processing, some of the signal components may be removed by adjusting the histogram when adjusting the luminance of the entire image. In addition, adjustment of processing parameters becomes complicated.

As described above, errors occur in each of the signal processing described above, leading to a decrease in accuracy. Therefore, when sequentially performing the multiple signal processing described above, errors are superimposed sequentially, and the loss amount of the information amount caused by the signal processing increases even further.

On the other hand, for some eye diseases, it is known that data obtained by performing Fourier transformation on the OCT image, which is in the same dimension as the interferogram, can be a diagnostic criterion (e.g., “Quantifying frequency content in cross-sectional retinal scans of diabetics vs. controls” described above). In this case, redundant processing must be performed in order to obtain useful information for diagnosis, resulting in reduced computational efficiency, decreased accuracy of processing results, and longer computation times.

Furthermore, in the OCT apparatus devices (OCT apparatuses), the software for generating OCT images differs for each model (type) of device, and the information lost in the processing of forming OCT images also differs for each model. As a result, the OCT analysis results obtained by performing information processing using the learned model that uses OCT images as input data also differ from model to model. This means that when OCT devices differ in model, OCT analysis results of the OCT devices cannot be compared. Therefore, in this case, the OCT analysis results cannot be systematically acquired.

In order to compensate for the differences between models of the OCT devices, it is necessary to collect and analyze a large amount of data. Thus, for example, it is virtually impossible to compensate for such differences in models of the OCT devices that were not widely available immediately after the start of sales, or in models of the OCT devices produced in small quantities for research or special applications. This make it difficult to obtain analysis results necessary for making important medical decisions.

In contrast, according to the embodiments, the reflection intensity distribution can be obtained from the interferogram(s) without performing each of the conventional signal processing described above. Alternatively, according to the embodiments, the analysis results (including statistical information) that would conventionally be obtained by performing image processing on the OCT image can be directly acquired from the interferogram(s) without performing each of the conventional signal processing described above.

This makes it possible to acquire the medical service supporting information including the OCT analysis result(s) and/or the OCT image(s) as the reflection intensity distribution, at high speed and with high accuracy without using advanced software technology. As a result, the analysis results of the eye of the examinee can be acquired with good accuracy and precision, or can be acquired efficiently with high accuracy. Further, since the OCT analysis result(s) can be systematically acquired from the interferograms, the OCT analysis result(s) can be acquired systematically even when the models of OCT devices differ.

Furthermore, according to the embodiments, it is particularly useful for applications that require high speed, such as preview, for example. Examples of such applications include generation of the OCTA images and removal of projection artifacts.

Furthermore, according to the embodiments, it is also useful in home OCT where the OCT apparatus is installed in the home of the examinee for the purpose of home medical care/remote medical care. For example, by extracting alone changes in the interferograms (or images formed based on the interferograms) while maintaining the interrelationship between the OCT apparatus and the home OCT, it can be used as a prognostic management monitoring tool for age-related macular degeneration or the like. In this case, the interferograms may be stored alone, and the functions of the ophthalmic information processing apparatus according to the embodiments may be realized by a server apparatus installed in a cloud or the like.

Furthermore, since the interferograms are stored alone, the weight of apparatuses capable of outputting the medical service supporting information can be reduced. In the case of performing arithmetic processing equivalent to a part of the generation processing of the medical service supporting information, the weight of the apparatus can be further reduced by performing the processing in the server apparatus installed in the cloud, etc. In this case, the server apparatus may be configured to perform generation processing of the medical service supporting information using the analysis model.

Furthermore, since the OCT analysis results can be systematically acquired regardless of differences in models of the OCT devices, the results can be compared with past OCT analysis results, enabling highly accurate analysis and observation when applied to progression analysis and follow-up.

Furthermore, according to the embodiments, at least one effect of the following five points may be obtained. First, software development becomes unnecessary when developing the OCT apparatus that acquires OCT images alone. Second, the technology related to OCT is useful for licensing to non-specialized research and development organizations and universities. Third, even when the details of the OCT configuration of competitors are unknown, OCT images using the interferograms from OCT of the competitors can be constructed and/or the OCT analysis results can be compared with that of the competitors. Fourth, by storing the interferograms, it will be possible to use them for purposes other than obtaining OCT analysis results in the future. Fifth, statistical processing can be performed utilizing the interferograms obtained in the past or the OCT analysis results obtained in the past.

The configuration according to the embodiments is not limited to the configuration according to the embodiments described above. Hereinafter, modification examples of the embodiments will be described focusing on the differences from the embodiments.

### First Modification Example

In the embodiments described above, a case in which the analysis model outputs the medical service supporting information including at least one of the OCT analysis results or the OCT image from the one or more interferograms has been mainly described. However, the configuration according to the embodiments is not limited to this. For example, the analysis model may be configured to output the medical service supporting information including the OCT analysis result(s) alone from the one or more interferograms. In this case, the analysis result information may be generated using the OCT image(s) formed from the interferograms, which are input data of the analysis model.

Hereinafter, the first modification example of the embodiments will be described focusing on the differences from the embodiments.

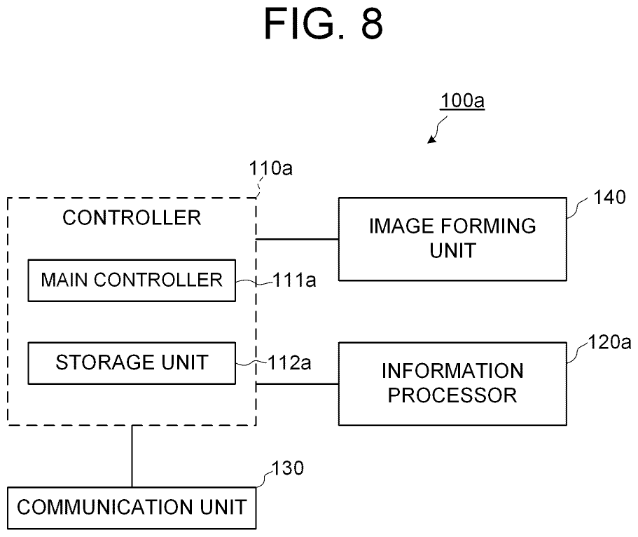

FIG. 8 shows a block diagram of an example of the configuration of the ophthalmic information processing apparatus according to the first modification example of the embodiments. In FIG. 8, like reference numerals designate like parts as in FIG. 3, and the redundant explanation may be omitted as appropriate.

The ophthalmic system 1 shown in FIG. 1 can include an ophthalmic information processing apparatus 100a according to the present modification example, instead of the ophthalmic information processing apparatus 100.

The configuration of the ophthalmic information processing apparatus 100a according to the present modification example differs from that of the ophthalmic information processing apparatus 100 mainly in that a controller 110a and an information processor 120a are provided instead of the controller 110 and the information processor 120, and that an image forming unit 140 is added.

The main difference between the controller 110a and the controller 110 is that the controller 110a controls the information processor 120a, the communication unit 130, and the image forming unit 140 instead of controlling the information processor 120 and the communication unit 130. The controller 110a includes a main controller 111a and a storage unit 112a.

The main difference between the main controller 111a and the main controller 111 is that the main controller 111a controls the information processor 120a and the image forming unit 140 instead of controlling the information processor 120. The difference between the storage unit 112a and the storage unit 112 is that the storage unit 112a stores computer program executed by the main controller 111a instead of computer program executed by the main controller 111.

The image forming unit 140 forms image data of the OCT image of the eye of the examinee, based on the one or more interferograms from the OCT apparatus 10. The image forming unit 140 can form the image data of the OCT image by performing signal processing similar to the conventional OCT image formation processing. Examples of the OCT image include an A-scan image, a B-scan image, a C-scan image, a projection image, an en-face image, a shadowgram, an OCT angiography (OCTA) image, a three-dimensional OCT image, and a tomographic image representing the morphology of the tomographic structures in a desired cross-sectional direction.

For example, the image forming unit 140 can form one or more A-scan images based on the one or more interferograms, and can form the B-scan image by arranging the formed one or more A-scan images.

For example, the image forming unit 140 can perform known image processing such as interpolation for interpolating pixels between B-scan images, and can form the image data of the three-dimensional OCT image. It should be noted that the image data of the three-dimensional OCT image means image data in which the positions of pixels are defined in a three-dimensional coordinate system. Examples of the image data of the three-dimensional OCT image include image data defined by voxels three-dimensionally arranged. Such image data is referred to as volume data or voxel data. In case of displaying an image based on the volume data, the image forming unit 140 performs rendering processing (e.g., volume rendering, maximum intensity projection (MIP), etc.) on the volume data. Thereby, the image data of a pseudo three-dimensional OCT image taken from a specific view direction can be formed.

Further, stack data of a plurality of B-scan images can also be formed as the image data of the three-dimensional OCT image. The stack data is image data formed by three-dimensionally arranging a plurality of B-scan images acquired along a plurality of scan lines based on positional relationship of the scan lines. That is, the stack data is image data obtained by representing the B-scan images, which are originally defined in their respective two-dimensional coordinate systems, by a single three-dimensional coordinate system. That is, the stack data is image data formed by embedding the B-scan images into a single three-dimensional space.

For example, the image forming unit 140 performs various types of rendering on the acquired three-dimensional data set (volume data, stack data, etc.). Thereby, a B-mode image (longitudinal cross-sectional image, axial cross-sectional image) in an arbitrary cross section, a C-mode image (transverse section image, horizontal cross-sectional image) in an arbitrary cross section, a projection image, a shadowgram, or the like is formed. An image in an arbitrary cross section such as a B-mode image or a C-mode image is formed by selecting pixels (voxels) on a designated cross section from the three-dimensional data set. The projection image is formed by projecting the three-dimensional data set in a predetermined direction (z direction, depth direction, axial direction). The shadowgram is formed by projecting a part of the three-dimensional data set in a predetermined direction. Examples of the part of the three-dimensional data set include partial data corresponding to a specific layer. An image having a viewpoint on the front side of the eye of the examinee, such as the C-mode image, the projection image, and the shadowgram, is called an en-face image (front image).

For example, the image forming unit 140 can build (form) the B-scan image or the front image (blood vessel emphasized image, angiogram) in which retinal blood vessels and choroidal blood vessels are emphasized (highlighted), based on the two or more interferograms acquired in time series. For example, the two or more interferograms in time series can be acquired by repeatedly scanning substantially the same site of the eye of the examinee.

In some embodiments, the image forming unit 140 compares the B-scan images in time series acquired by B-scan for substantially the same site, converts the pixel value of a change portion of the signal intensity into a pixel value corresponding to the change portion, and builds the emphasized image in which the change portion is emphasized. Further, the image forming unit 140 forms an OCTA image by extracting information of a predetermined thickness at a desired site from a plurality of built emphasized images and building as an en-face image.

The information processor 120a has the similar configuration to the information processor 120. The main difference between the information processor 120a and the information processor 120 is that the information processor 120a uses the analysis model configured to input the one or more interferograms and to output information excluding OCT images among the medical service supporting information.

In other words, the analysis model building unit in the information processor 120a performs machine learning on a learning model, the learning model using the one or more interferograms as input and outputting the information excluding the OCT images among the medical service supporting information described above. Thereby, the analysis model is built as the learned model. The analysis model building unit according to the present modification example can build the analysis model according to the present modification example in accordance with the flow shown in FIG. 5, in the same manner as the analysis model building unit 200 according to the embodiments. The analyzer in the information processor 120a generates the medical service supporting information excluding the OCT images from the acquired one or more interferograms, using the analysis model previously built by the analysis model building unit. The analysis result information generator in the information processor 120a generates the analysis result information including the OCT image formed by the image forming unit 140 and the medical service supporting information, which is acquired by the analyzer, excluding the OCT image. The controller 110a (main controller 111a) can cause the analysis result information generated by the analysis result information generator to be displayed on the display apparatus 190.

In the present modification example, each of the controller 110a, the information processor 120a, and the image forming unit 140 includes one or more processors. The functions of the information processor 120a are realized by one or more information processing processors. The functions of the image forming unit 140 is realized by one or more image forming processor. In some embodiments, the functions of the controller 110a, the information processor 120a, and the image forming unit 140 are realized by two processors or a single processor.

The OCT image formed by the image forming unit 140 is an example of the “second OCT image” according to the embodiments.

### [Operation Example]

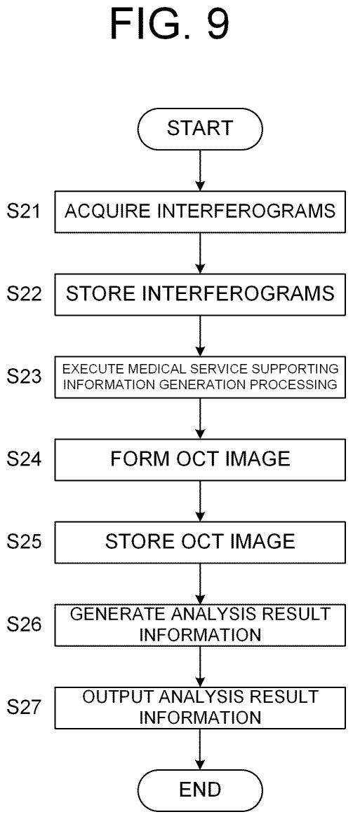

FIG. 9 shows a flowchart of an example of an operation of the ophthalmic information processing apparatus 100a according to the present modification example. The storage unit 112 or a storage unit (not shown) in the information processor 120a stores a computer program for realizing the processing shown in FIG. 9. One or more processors configured to realize the functions of the ophthalmic information processing apparatus 100a operates according to the computer program, and thereby the one or more processors perform the processing shown in FIG. 9.

In FIG. 9, as in FIG. 6, it is assumed that the data set of the analysis model is expanded in advance in the working memory of the information processor 120a, the data set being read out from the storage unit 112a or the storage unit (not shown) in the information processor 120a.

### (S21: Acquire Interferograms)

First, the main controller 111a controls the communication unit 130 to establish the communication connection with the communication unit 13 in the OCT apparatus 10, in the same manner as in step S11.

The main controller iiia acquires the one or more interferograms stored in the OCT apparatus 10 via the communication path established with the communication unit 13 in the OCT apparatus 10.

### (S22: Store Interferograms)

Subsequently, the main controller 111a stores the one or more interferograms acquired in step S21 in the storage unit 112a, in the same manner as in step S12.

### (S23: Execute Medical Service Supporting Information Generation Processing)

Subsequently, the main controller 111a controls the analyzer in the information processor 120a to execute the medical service supporting information generation processing, using the one or more interferograms stored in step S22 as input data.

The analyzer according to the present modification example outputs the medical service supporting information including the OCT analysis results as the output data, by inputting the one or more interferograms to the data set of the analysis model expanded in advance in the working memory. In the present modification example, the output medical service supporting information does not include the OCT images.

### (S24: Form OCT Image)

Subsequently, the main controller 111a controls the image forming unit 140 to form the OCT image based on the one or more interferograms stored in step S22.

### (S25: Store OCT Image)

Subsequently, the main controller 111a stores the image data of the OCT image formed in step S24 in the storage unit 112a.

In the present modification example, the processing of step S23 may be performed after the processing of steps S24 to S25.

### (S26: Generate Analysis Result Information)

Next, the main controller 111a controls the analysis result information generator in the information processor 120a to generate the analysis result information of the eye of the examinee using the medical service supporting information obtained in step S23 and the OCT image stored in step S25.

### (S27: Output Analysis Result Information)

Next, the main controller 111a outputs the analysis result information generated in step S26, in the same manner as in step S15. For example, the main controller 111a controls the display apparatus 190 to display the analysis result information generated in step S26, as a display controller. For example, the main controller 111a controls a printing device not shown to print the analysis result information generated in step S26, as an output controller. In some embodiments, the main controller 111a stores the analysis result information generated in step S26 in an external server device (not shown) in association with an identification number of the examinee.

This terminates the processing of the ophthalmic information processing apparatus 100a (END).

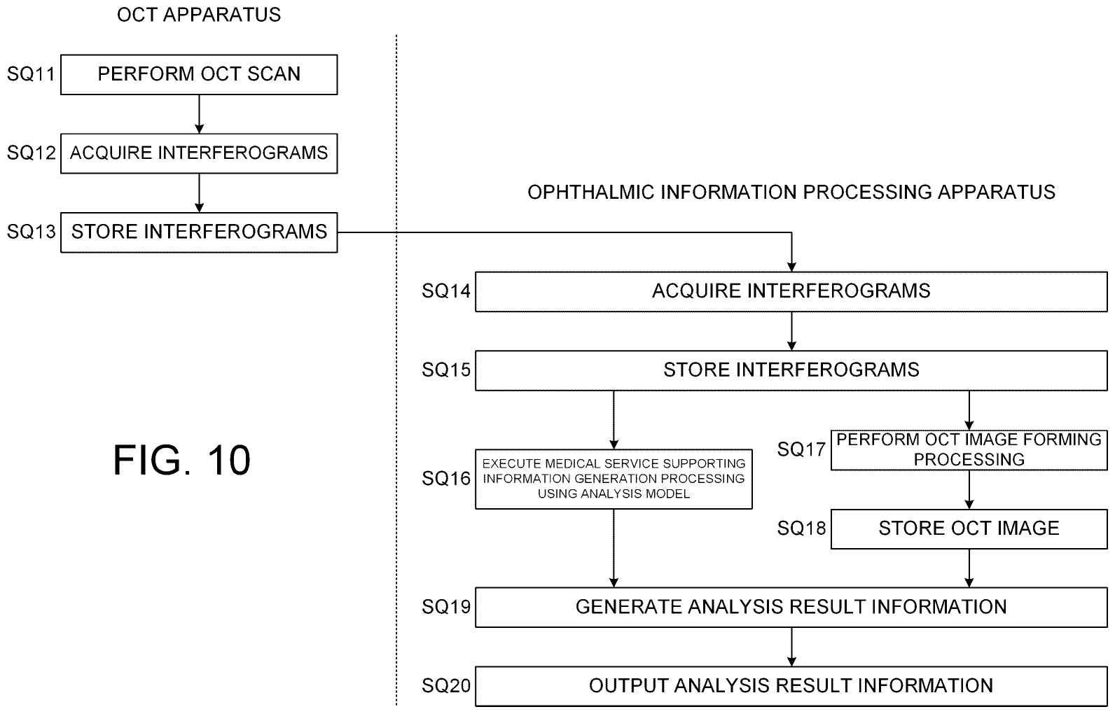

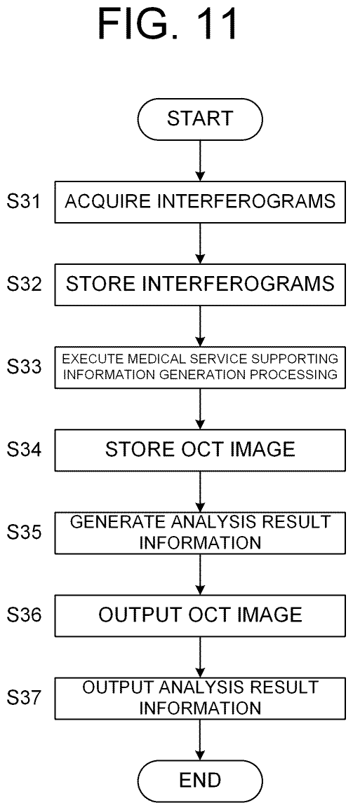

FIG. 10 shows an example of an operation sequence of the ophthalmic system 1 according to the first modification example of the embodiments. In FIG. 10, it is assumed that the analysis model has already been built by the analysis model building unit and the data set of the analysis model has been stored in the storage unit 112a or the storage unit (not shown) in the information processor 120a, in the same manner as in FIG. 7.

In the OCT apparatus 10, the controller 11 controls the OCT optical system 12 to perform OCT scan on the eye of the examinee under photographing conditions (photographing light quantity, photographing region) set in advance (SQ11).

The controller 11 acquires the one or more interferograms by sequentially acquiring the detection results of the interference light acquired by the OCT scan (SQ12), and stores the acquired one or more interferograms in the storage unit (not shown) in the controller 11 (SQ13).

In the ophthalmic information processing apparatus 100a, the controller 110a controls the communication unit 130 to establish the communication connection with the OCT apparatus 10 to acquire the one or more interferograms from the OCT apparatus 10 (SQ14). After the one or more interferograms have been acquired by the OCT apparatus 10, the controller 11 may control the communication unit 13 to establish the communication connection with the ophthalmic information processing apparatus 100a and to transmit the one or more interferograms to the ophthalmic information processing apparatus 100a.

The controller 110a stores the one or more interferograms acquired from the OCT apparatus 10 in the storage unit 112a or the storage unit in the information processor 120a (SQ15).

Subsequently, the controller 110a reads out the one or more interferograms, and controls the analyzer to execute the medical service supporting information generation processing using the one or more interferograms as input data (SQ16). The data set of the analysis model has been read out from the storage unit 112a or the storage unit (not shown) in the information processor 120a, and has already been expanded in the working memory. The analyzer outputs the medical service supporting information including the OCT analysis results as the output data, by inputting the one or more interferograms to the data set of the analysis model expanded in the working memory.

On the other hand, the controller 110a controls the image forming unit 140 to form the OCT image based on the one or more interferograms read out from the storage unit 112a or the storage unit (not shown) in the information processor 120a (SQ17). The controller 110a stores the image data of the formed OCT image in the storage unit 112a (SQ18).

The controller 110a controls the analysis result information generator to generate the analysis result information using the medical service supporting information output from the analysis model and the OCT image formed by the image forming unit 140 (SQ19). After that, the controller 110a controls the display apparatus 190, etc. to output the generated analysis result information (SQ20).

As explained above, according to the present modification example, in the same manner as in the embodiments, the medical service supporting information including the OCT analysis result(s) is generated directly from the one or more interferograms using the analysis model obtained by machine learning. This eliminates the need for complex signal processing on the acquired interferograms. As a result, when acquiring the OCT analysis result(s), the loss of information amount accompanying signal processing can be suppressed, and the analysis result of the eye of the examinee can be obtained with good accuracy and precision, or with high precision and efficiency. In addition, according to the present modification example, the same effects as those described above can be obtained in the same manner as in the embodiments.

### Second Modification Example

In the embodiments, a case in which the medical service supporting information including at least one of the OCT analysis result(s) or the OCT image is acquired from the one or more interferograms using the analysis model, and the analysis result information including the acquired medical service supporting information is generated has been mainly described. However, the configuration according to the embodiments is not limited to this. For example, in case that the OCT analysis result(s) and the OCT image are acquired from the one or more interferograms using the analysis model, the configuration of the embodiments may be configured to output the acquired OCT image separately.

Hereinafter, the second modification example of the embodiments will be described focusing on the differences from the embodiments.

The configuration of the ophthalmic information processing apparatus according to the second modification example of the embodiments is similar to that of the ophthalmic information processing apparatus 100 according to the embodiments. The ophthalmic system 1 shown in FIG. 1 can include an ophthalmic information processing apparatus according to the present modification example, instead of the ophthalmic information processing apparatus 100.

The main difference between the operation of the ophthalmic information processing apparatus according to the present modification example and that of the ophthalmic information processing apparatus 100 is that the ophthalmic information processing apparatus according to the present modification example generates the OCT image and the OCT analysis result(s) as the medical service supporting information and outputs the generated OCT image to the display apparatus 190, etc. separately from the OCT analysis results.

### [Operation Example]

FIG. 11 shows a flowchart of an example of an operation of the ophthalmic information processing apparatus according to the present modification example. The storage unit according to the present modification example or a storage unit (not shown) in the information processor stores a computer program for realizing the processing shown in FIG. 11. One or more processors configured to realize the functions of the ophthalmic information processing apparatus according to the present modification example operates according to the computer program, and thereby the one or more processors perform the processing shown in FIG. 11.

In FIG. 11, it is assumed that the data set of the analysis model is expanded in advance in the working memory of the information processor, the data set being read out from the storage unit or the storage unit (not shown) in the information processor, in the same manner as in FIG. 6.

### (S31: Acquire Interferograms)

First, the main controller according to the present modification example controls the communication unit 130 to establish the communication connection with the communication unit 13 in the OCT apparatus 10, in the same manner as in step S11.

The main controller acquires the one or more interferograms stored in the OCT apparatus 10 via the communication path established with the communication unit 13 in the OCT apparatus 10.

### (S32: Store Interferograms)

Subsequently, the main controller stores the one or more interferograms acquired in step S31 in the storage unit, in the same manner as in step S12.

### (S33: Execute Medical Service Supporting Information Generation Processing)

Subsequently, the main controller controls the analyzer in the information processor according to the present modification example to execute the medical service supporting information generation processing, using the one or more interferograms stored in step S32 as input data.

The analyzer according to the present modification example outputs the medical service supporting information including the OCT analysis results and the OCT image as the output data, by inputting the one or more interferograms to the data set of the analysis model expanded in advance in the working memory.

### (S34: Store OCT Image)

Subsequently, the main controller stores the OCT image acquired in the medical service supporting information generation processing in step S33 in the storage unit or the storage unit (not shown) in the information processor, separately from the OCT analysis result(s).

### (S35: Generate Analysis Result Information)

Next, the main controller controls the analysis result information generator in the information processor according to the present modification example to generate the analysis result information of the eye of the examinee, using some or all of the medical service supporting information acquired in step S33.

### (S36: Output OCT Image)

Next, the main controller outputs the OCT image stored in step S34. For example, the main controller controls the display apparatus 190 to display the OCT image stored in step S34, as a display controller. For example, the main controller controls a printing device not shown to print the OCT image stored in step S34, as an output controller. In some embodiments, the main controller stores the OCT image stored in step S34 in an external server device (not shown) in association with an identification number of the examinee.

### (S37: Output Analysis Result Information)

Next, the main controller outputs the analysis result information generated in step S35, in the same manner as in step S15. For example, the main controller controls the display apparatus 190 to display the analysis result information generated in step S35, as a display controller. For example, the main controller controls a printing device not shown to print the analysis result information generated in step S35, as an output controller. In some embodiments, the main controller stores the analysis result information generated in step S35 in an external server device (not shown) in association with an identification number of the examinee.

This terminates the processing of the ophthalmic information processing apparatus according to the present modification example (END).

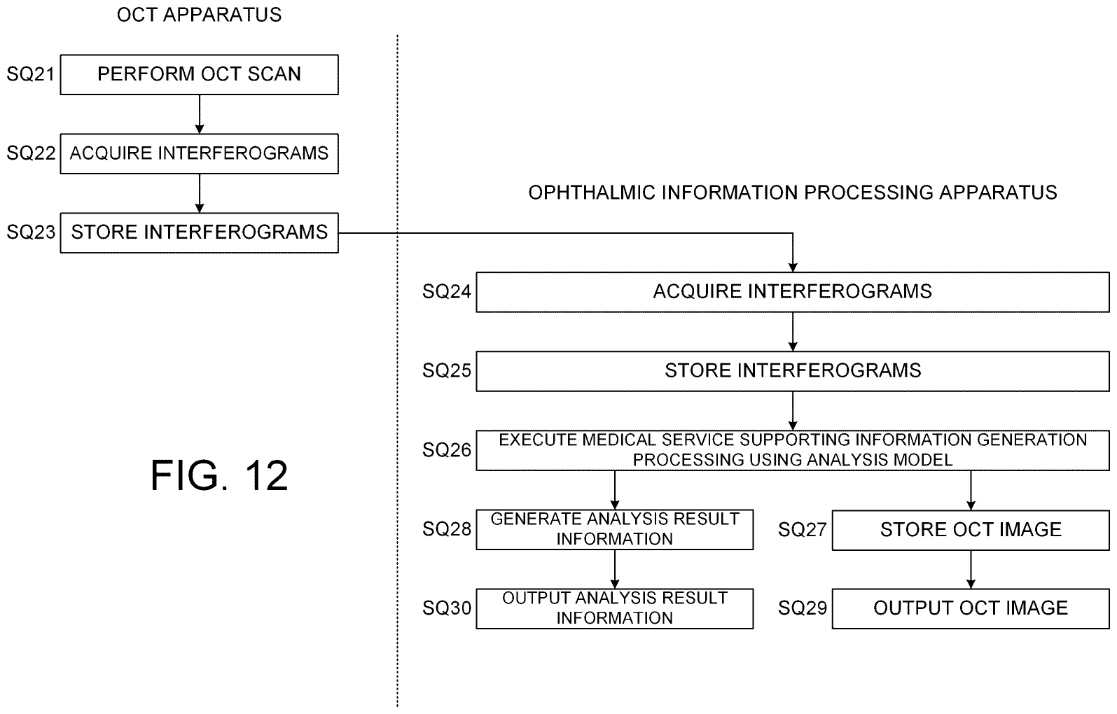

FIG. 12 shows an example of an operation sequence of the ophthalmic system 1 according to the second modification example of the embodiments. In FIG. 12, it is assumed that the analysis model has already been built by the analysis model building unit and the data set of the analysis model has been stored in the storage unit according to the present modification example or the storage unit (not shown) in the information processor according to the present modification example, in the same manner as in FIG. 7.

In the OCT apparatus 10, the controller 11 controls the OCT optical system 12 to perform OCT scan on the eye of the examinee under photographing conditions (photographing light quantity, photographing region) set in advance (SQ21).

The controller 11 acquires the one or more interferograms by sequentially acquiring the detection results of the interference light acquired by the OCT scan (SQ22), and stores the acquired one or more interferograms in the storage unit (not shown) in the controller 11 (SQ23).

In the ophthalmic information processing apparatus according to the present modification example, the controller controls the communication unit to establish the communication connection with the OCT apparatus 10 to acquire the one or more interferograms from the OCT apparatus 10 (SQ24). After the one or more interferograms have been acquired by the OCT apparatus 10, the controller may control the communication unit to establish the communication connection with the ophthalmic information processing apparatus according to the present modification example and to transmit the one or more interferograms to the ophthalmic information processing apparatus.

The controller in the ophthalmic information processing apparatus according to the present modification example stores the one or more interferograms acquired from the OCT apparatus in the storage unit according to the present modification example or the storage unit (not shown) in the information processor according to the present modification example (SQ25).

Subsequently, the controller reads out the one or more interferograms, and controls the analyzer to execute the medical service supporting information generation processing using the one or more interferograms as input data (SQ26). The data set of the analysis model has been read out from the storage unit or the storage unit (not shown) in the information processor, and has already been expanded in the working memory. The analyzer outputs the medical service supporting information including the OCT analysis results and the OCT image as the output data, by inputting the one or more interferograms to the data set of the analysis model expanded in the working memory.

The controller stores the OCT image output from the analysis model in the storage unit according to the present modification example (SQ27). After that, the controller controls the display apparatus 190, etc. to output the OCT image stored in the storage unit (SQ29).

On the other hand, the controller controls the analysis result information generator to generate the analysis result information using the medical service supporting information including the OCT analysis result(s) output from the analysis model and the OCT image (SQ28). After that, the controller controls the display apparatus 190, etc. to output the generated analysis result information (SQ30).

As explained above, according to the present modification example, in the same manner as in the embodiments, the medical service supporting information including the OCT analysis result(s) and the OCT image is generated directly from the one or more interferograms using the analysis model obtained by machine learning. This eliminates the need for complex signal processing on the acquired interferograms. As a result, the loss of information amount accompanying signal processing can be suppressed, and the analysis result of the eye of the examinee can be obtained with good accuracy and precision, or with high precision and efficiency. In addition, according to the present modification example, the same effects as those described above can be obtained in the same manner as in the embodiments.

### Third Modification Example

In the embodiments described above or the modification examples thereof, a case in which the medical service supporting information is output using the analysis model with the one or more interferograms as input data has been mainly described. However, the configuration according to the embodiments is not limited to this. For example, the configuration of the embodiments may be configured to output the medical service supporting information including the OCT analysis result(s) from the analysis model with the one or more interferograms and the OCT image(s) as input data.

Hereinafter, the third modification example of the embodiments will be described focusing on the differences from the first modification example of the embodiments.

The configuration of the ophthalmic information processing apparatus according to the third modification example of the embodiments is similar to that of the ophthalmic information processing apparatus 100a according to the first modification example of the embodiments.

It should be noted that the analysis model building unit according to the present modification example performs machine learning using the one or more interferograms and the OCT image(s) as input, and builds the analysis model configured to output the medical service supporting information. For example, the analysis model building unit can build the analysis model that can output the medical service supporting information by performing machine learning on the learning model using the teaching data. In this case, the teaching data may be data of a plurality of pairs of input data and output data. Here, the input data are the one or more interferograms and the OCT image(s). The output data are the medical service supporting information according to the input data. Here, the input data includes the one or more interferograms and the OCT image(s) based on the one or more interferograms. The medical service supporting information may not include the OCT image(s). Alternatively, the medical service supporting information may include the OCT image in which a site related to a disease (site of lesion, site of interest) is identifiably depicted, or the OCT image with higher image quality than the OCT image for the input data.

The ophthalmic system 1 shown in FIG. 1 can include an ophthalmic information processing apparatus according to the present modification example, instead of the ophthalmic information processing apparatus 100.

The main difference between the operation of the ophthalmic information processing apparatus according to the present modification example and that of the ophthalmic information processing apparatus 100a is that the ophthalmic information processing apparatus according to the present modification example uses the analysis model with the one or more interferograms and the OCT image formed based on the one or more interferograms as input data.

### [Operation Example]

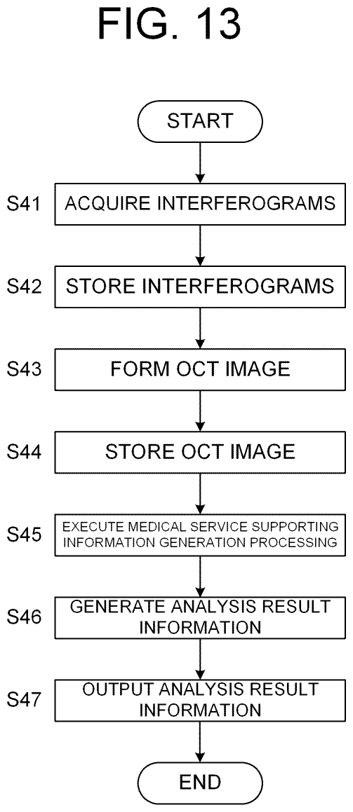

FIG. 13 shows a flowchart of an example of an operation of the ophthalmic information processing apparatus according to the present modification example. The storage unit according to the present modification example or a storage unit (not shown) in the information processor stores a computer program for realizing the processing shown in FIG. 13. One or more processors configured to realize the functions of the ophthalmic information processing apparatus according to the present modification example operates according to the computer program, and thereby the one or more processors perform the processing shown in FIG. 13.

In FIG. 13, it is assumed that the data set of the analysis model is expanded in advance in the working memory of the information processor, the data set being read out from the storage unit or the storage unit (not shown) in the information processor, in the same manner as in FIG. 6.

### (S41: Acquire Interferograms)

First, the main controller according to the present modification example controls the communication unit 130 to establish the communication connection with the communication unit 13 in the OCT apparatus 10, in the same manner as in step S11.

The main controller acquires the one or more interferograms stored in the OCT apparatus 10 via the communication path established with the communication unit 13 in the OCT apparatus 10.

### (S42: Store Interferograms)

Subsequently, the main controller stores the one or more interferograms acquired in step S41 in the storage unit, in the same manner as in step S12.

### (S43: Form OCT Image)

Subsequently, the main controller controls the image forming unit 140 to form the OCT image based on the one or more interferograms stored in step S42.

(S44: Store OCT Image) Subsequently, the main controller stores the image data of the OCT image formed in step S43 in the storage unit.

### (S45: Execute Medical Service Supporting Information Generation Processing)

Subsequently, the main controller controls the analyzer in the information processor to execute the medical service supporting information generation processing using the analysis model with the one or more interferograms stored in step S42 and the OCT image(s) stored in step S44 as input data.

The analyzer outputs the medical service supporting information described above as the output data, by inputting the one or more interferograms and the OCT image(s) to the data set of the analysis model expanded in advance in the working memory.

### (S46: Generate Analysis Result Information)

Next, the main controller controls the analysis result information generator in the information processor to generate the analysis result information of the eye of the examinee using the medical service supporting information obtained in step S45.

The analysis result information generator generates the analysis result information including at least a part of the medical service supporting information, as described above.

### (S47: Output Analysis Result Information)

Next, the main controller outputs the analysis result information generated in step S46. For example, the main controller controls the display apparatus 190 to display the analysis result information generated in step S46, as a display controller. For example, the main controller controls a printing device not shown to print the analysis result information generated in step S46, as an output controller. In some embodiments, the main controller stores the analysis result information generated in step S46 in an external server device (not shown) in association with an identification number of the examinee.

This terminates the processing of the ophthalmic information processing apparatus according to the present modification example (END).

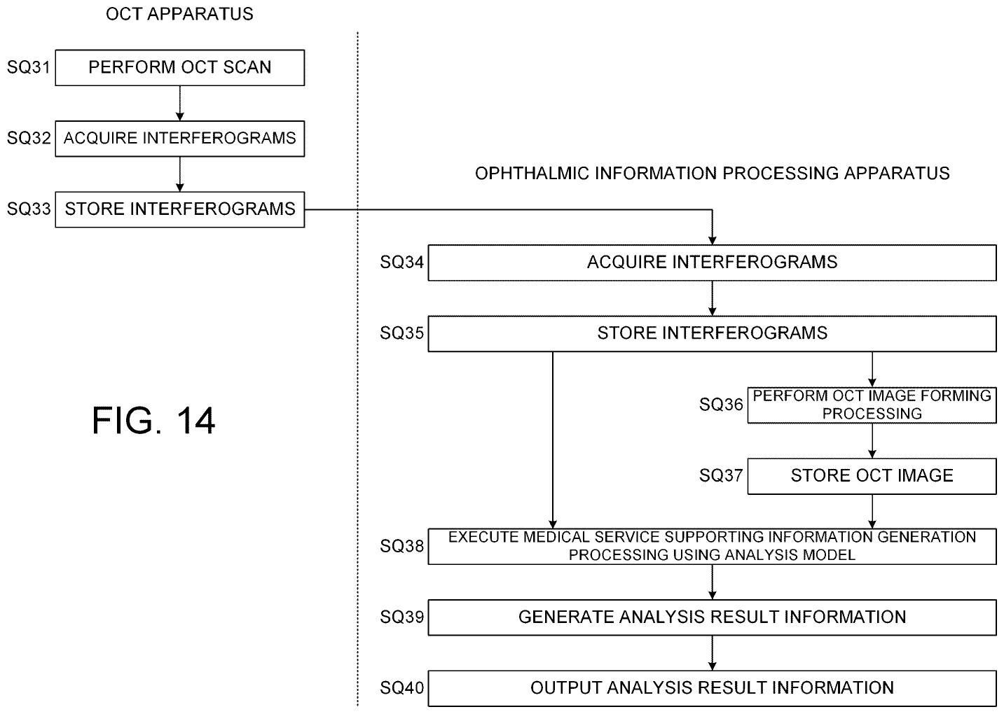

FIG. 14 shows an example of an operation sequence of the ophthalmic system 1 according to the third modification example of the embodiments. In FIG. 14, it is assumed that the analysis model has already been built by the analysis model building unit according to the present modification example and the data set of the analysis model has been stored in the storage unit according to the present modification example or the storage unit (not shown) in the information processor according to the present modification example, in the same manner as in FIG. 7.

In the OCT apparatus 10, the controller 11 controls the OCT optical system 12 to perform OCT scan on the eye of the examinee under photographing conditions (photographing light quantity, photographing region) set in advance (SQ31).

The controller 11 acquires the one or more interferograms by sequentially acquiring the detection results of the interference light acquired by the OCT scan (SQ32), and stores the acquired one or more interferograms in the storage unit (not shown) in the controller 11 (SQ33).

In the ophthalmic information processing apparatus according to the present modification example, the controller controls the communication unit to establish the communication connection with the OCT apparatus 10 to acquire the one or more interferograms from the OCT apparatus 10 (SQ34). After the one or more interferograms have been acquired by the OCT apparatus 10, the controller 11 may control the communication unit 13 to establish the communication connection with the ophthalmic information processing apparatus according to the present modification example and to transmit the one or more interferograms to the ophthalmic information processing apparatus.

The controller in the ophthalmic information processing apparatus according to the present modification example stores the one or more interferograms acquired from the OCT apparatus 10 in the storage unit according to the present modification example or the storage unit (not shown) in the information processor according to the present modification example (SQ35).

Subsequently, the controller controls the image forming unit according to the present modification example to form the OCT image based on the one or more interferograms read out from the storage unit or the storage unit (not shown) in the information processor (SQ36). The controller stores the image data of the formed OCT image in the storage unit or the storage unit (not shown) in the information processor (SQ37).

On the other hand, the controller reads out the one or more interferograms and the image data of the OCT image(s), and controls the analyzer according to the present modification example to execute the medical service supporting information generation processing using the one or more interferograms and the OCT image(s) as input data (SQ38). The data set of the analysis model has been read out from the storage unit or the storage unit (not shown) in the information processor, and has already been expanded in the working memory. The analyzer outputs the medical service supporting information described above as the output data, by inputting the one or more interferograms and the OCT image(s) to the data set of the analysis model expanded in advance in the working memory.

The controller controls the analysis result information generator to generate the analysis result information using at least a part of the medical service supporting information output from the analysis model (SQ39). After that, the controller controls the display apparatus 190, etc. to output the generated analysis result information (SQ40).

As explained above, according to the present modification example, in the same manner as in the embodiments, the medical service supporting information is generated directly from the one or more interferograms and the OCT image(s) using the analysis model obtained by machine learning. Thereby, the OCT analysis result(s) can be acquired without performing complex signal processing on the acquired interferograms. As a result, the loss of information amount accompanying signal processing can be suppressed, and the analysis result of the eye of the examinee can be obtained with good accuracy and precision, or with high precision and efficiency. In addition, according to the present modification example, the same effects as those described above can be obtained in the same manner as in the embodiments.

### Fourth Modification Example

The configuration according to the third modification example of the embodiment is configured to form the OCT image from the one or more interferograms, and to output the medical service supporting information by inputting the formed OCT images together with the one or more interferograms into the analysis model. However, the configuration according to the embodiments is not limited to this. For example, the formed OCT image may be input to the analysis model and may be displayed on the display apparatus 190, etc.

Hereinafter, the fourth modification example of the embodiments will be described focusing on the differences from the third modification example of the embodiments.

The configuration of the ophthalmic information processing apparatus according to the fourth modification example of the embodiments is similar to that of the ophthalmic information processing apparatus according to the third modification example of the embodiments (i.e., ophthalmic information processing apparatus 100a according to the first modification example). The ophthalmic system 1 shown in FIG. 1 can include an ophthalmic information processing apparatus according to the present modification example, instead of the ophthalmic information processing apparatus 100.

The main difference between the operation of the ophthalmic information processing apparatus according to the present modification example and that of the ophthalmic information processing apparatus according to the third modification example is that the OCT image is input into the analysis model together with the one or more interferograms and is displayed on the display apparatus 190, etc.

### [Operation Example]

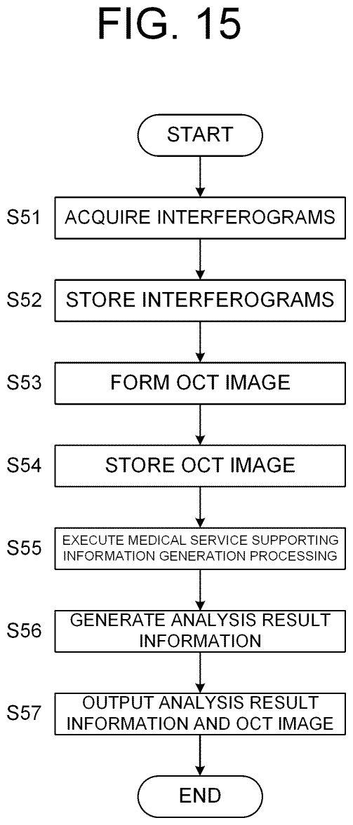

FIG. 15 shows a flowchart of an example of an operation of the ophthalmic information processing apparatus according to the present modification example. The storage unit according to the present modification example or a storage unit (not shown) in the information processor stores a computer program for realizing the processing shown in FIG. 15. One or more processors configured to realize the functions of the ophthalmic information processing apparatus according to the present modification example operates according to the computer program, and thereby the one or more processors perform the processing shown in FIG. 15.

In FIG. 15, it is assumed that the data set of the analysis model is expanded in advance in the working memory of the information processor, the data set being read out from the storage unit or the storage unit (not shown) in the information processor, in the same manner as in FIG. 13.

### (S51: Acquire Interferograms)

First, the main controller according to the present modification example controls the communication unit 130 to establish the communication connection with the communication unit 13 in the OCT apparatus 10, in the same manner as in step S41.

The main controller acquires the one or more interferograms stored in the OCT apparatus 10 via the communication path established with the communication unit 13 in the OCT apparatus 10.

### (S52: Store Interferograms)

Subsequently, the main controller stores the one or more interferograms acquired in step S51 in the storage unit, in the same manner as in step S42.

### (S53: Form OCT Image)

Subsequently, the main controller controls the image forming unit 140 to form the OCT image based on the one or more interferograms stored in step S52, in the same manner as in step S43.

### (S54: Store OCT Image)

Subsequently, the main controller stores the image data of the OCT image formed in step S53 in the storage unit, in the same manner as in step S44.

### (S55: Execute Medical Service Supporting Information Generation Processing)

Subsequently, the main controller controls the analyzer in the information processor to execute the medical service supporting information generation processing using the analysis model, in the same manner as in step S45.

The analyzer outputs the medical service supporting information described above as the output data, by inputting the one or more interferograms and the OCT image(s) to the data set of the analysis model expanded in advance in the working memory.

### (S56: Generate Analysis Result Information)

Next, the main controller controls the analysis result information generator in the information processor to generate the analysis result information of the eye of the examinee using the medical service supporting information obtained in step S55.

The analysis result information generator generates the analysis result information including at least a part of the medical service supporting information, as described above.

### (S57: Output Analysis Result Information and OCT Image)

Next, the main controller outputs the analysis result information generated in step S56 and the OCT image(s) stored in step S54. For example, the main controller controls the display apparatus 190 to display the analysis result information generated in step S56 and the OCT image(s) stored in step S54, as a display controller. The main controller can cause the analysis result information and the OCT image(s) to be displayed on the same screen of the display apparatus 190. Alternatively, the main controller can cause the analysis result information and the OCT image(s) to be displayed on display apparatuses that are different from each other. For example, the main controller controls a printing device not shown to print the analysis result information generated in step S56 and the OCT image(s) stored in step S54, as an output controller. In some embodiments, the main controller stores the analysis result information generated in step S56 and the OCT image(s) stored in step S54 in an external server device (not shown) in association with an identification number of the examinee.

This terminates the processing of the ophthalmic information processing apparatus according to the present modification example (END).

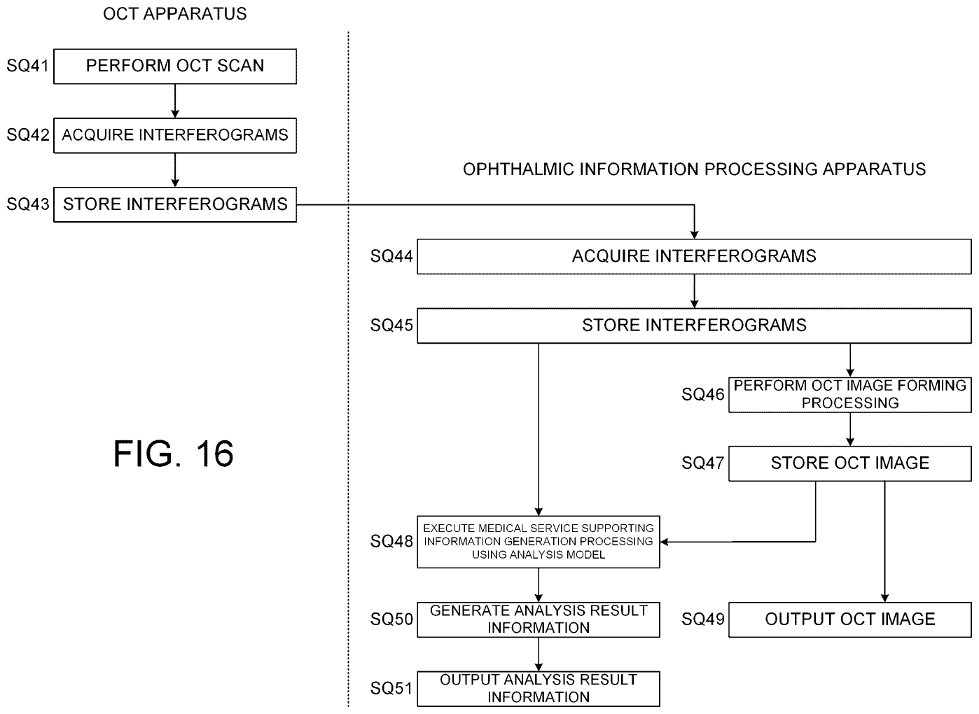

FIG. 16 shows an example of an operation sequence of the ophthalmic system 1 according to the fourth modification example of the embodiments. In FIG. 16, it is assumed that the analysis model has already been built by the analysis model building unit according to the present modification example and the data set of the analysis model has been stored in the storage unit according to the present modification example or the storage unit (not shown) in the information processor according to the present modification example, in the same manner as in FIG. 14.

In the OCT apparatus 10, the controller 11 controls the OCT optical system 12 to perform OCT scan on the eye of the examinee under photographing conditions (photographing light quantity, photographing region) set in advance (SQ41).

The controller 11 acquires the one or more interferograms by sequentially acquiring the detection results of the interference light acquired by the OCT scan (SQ42), and stores the acquired one or more interferograms in the storage unit (not shown) in the controller 11 (SQ43).

In the ophthalmic information processing apparatus according to the present modification example, the controller controls the communication unit to establish the communication connection with the OCT apparatus 10 to acquire the one or more interferograms from the OCT apparatus 10 (SQ44). After the one or more interferograms have been acquired by the OCT apparatus 10, the controller may control the communication unit to establish the communication connection with the ophthalmic information processing apparatus according to the present modification example and to transmit the one or more interferograms to the ophthalmic information processing apparatus.

The controller in the ophthalmic information processing apparatus according to the present modification example stores the one or more interferograms acquired from the OCT apparatus 10 in the storage unit according to the present modification example or the storage unit (not shown) in the information processor according to the present modification example (SQ45).

Subsequently, the controller controls the image forming unit according to the present modification example to form the OCT image based on the one or more interferograms read out from the storage unit or the storage unit (not shown) in the information processor (SQ46). The controller stores the image data of the formed OCT image in the storage unit or the storage unit (not shown) in the information processor (SQ47).

Subsequently, the controller reads out the one or more interferograms and the image data of the OCT image(s), and controls the analyzer according to the present modification example to execute the medical service supporting information generation processing using the one or more interferograms and the OCT image(s) as input data (SQ48). The data set of the analysis model has been read out from the storage unit or the storage unit (not shown) in the information processor, and has already been expanded in the working memory. The analyzer outputs the medical service supporting information described above as the output data, by inputting the one or more interferograms and the OCT image(s) to the data set of the analysis model expanded in advance in the working memory.

The controller controls the display apparatus 190, etc. to output the stored OCT image(s) (SQ49). On the other hand, the controller controls the analysis result information generator to generate the analysis result information using at least a part of the medical service supporting information output from the analysis model (SQ50). After that, the controller controls the display apparatus 190, etc. to output the generated analysis result information (SQ51).

As explained above, according to the present modification example, in the same manner as in the embodiments, the medical service supporting information is generated directly from the one or more interferograms and the OCT image(s) using the analysis model obtained by machine learning. Thereby, the OCT analysis result(s) can be acquired without performing complex signal processing on the acquired interferograms. As a result, the loss of information amount accompanying signal processing can be suppressed, and the analysis result of the eye of the examinee can be obtained with good accuracy and precision, or with high precision and efficiency. In addition, according to the present modification example, the same effects as those described above can be obtained in the same manner as in the embodiments.

### Fifth Modification Example

In the embodiments or the modification examples thereof, a case in which the medical service supporting information is output using the analysis model with the one or more interferograms, or the one or more interferograms and the OCT image(s) as input data has been mainly described. However, the configuration according to the embodiments is not limited to this. For example, the medical service supporting information may be output by inputting input data into the analysis model. Here, the input data is data to which background data of the examinee has been added to the one or more interferograms, or the one or more interferograms and the OCT image(s).

Hereinafter, the fifth modification example of the embodiments will be described focusing on the differences from the embodiments.

The configuration of the ophthalmic information processing apparatus according to the fifth modification example of the embodiments is similar to that of the ophthalmic information processing apparatus 100 according to the embodiments.

It should be noted that the analysis model building unit according to the present modification example performs machine learning using the one or more interferograms and the background data of the examinee as input, and builds the analysis model configured to output the medical service supporting information. For example, the analysis model building unit can build the analysis model that can output the medical service supporting information by performing machine learning on the learning model using the teaching data. In this case, the teaching data may be data of a plurality of pairs of input data and output data. Here, the input data are the one or more interferograms and the background data. The output data are the medical service supporting information according to the input data.

In some embodiments, the analysis model building unit performs machine learning using the one or more interferograms, the background data, and the OCT image(s) as input, and builds the analysis model configured to output the medical service supporting information. The OCT image is formed based on the one or more interferograms, which are one of the input data, by an image forming unit (for example, the image forming unit 140 shown in FIG. 8). For example, the analysis model building unit can build the analysis model that can output the medical service supporting information by performing machine learning on the learning model using the teaching data. In this case, the teaching data may be data of a plurality of pairs of input data and output data. Here, the input data are the one or more interferograms, the background data, and the OCT image(s). The output data are the medical service supporting information according to the input data.

Examples of the background data include a gender, an age, a visual acuity value, a refractive power value, an equivalent spherical power, an intraocular pressure value, a fundus front image, a fluorescein fundus angiography image, an anterior segment image, a visual field test result, a corneal curvature, a corneal thickness, an axial length, a history of present illness, a past medical history, a family medical history, a body height, weight, an applied medicine, a blood test result, and an urine test result. The fundus front image, and the fluorescence fundus angiography image can be acquired using the fundus camera. Examples of the visual field test result include a Mean Defect value and a Pattern Standard Deviation value. The background data may include gene information, a history of life, or a history of trauma. The history of life may include an occupational history, an early developmental history, activities of daily living (ADL) information, or a preferences (drinking, smoking). The activities of daily living information may be information that has been categorized in advance, such as the condition of the eye. Examples of the past medical history include history of visits to ophthalmologists, systemic diseases, and a physical symptom. The background data that are input to the analysis model may include at least one of the background data described above.

The ophthalmic system 1 shown in FIG. 1 can include an ophthalmic information processing apparatus according to the present modification example, instead of the ophthalmic information processing apparatus 100.

The main difference between the operation of the ophthalmic information processing apparatus according to the present modification example and that of the ophthalmic information processing apparatus 100 is that the ophthalmic information processing apparatus according to the present modification example uses the analysis model with the one or more interferograms and the background data of the examinee as input data. Alternatively, the main difference between the operation of the ophthalmic information processing apparatus according to the present modification example and that of the ophthalmic information processing apparatus 100 is that the ophthalmic information processing apparatus according to the present modification example uses the analysis model with the one or more interferograms, the OCT image, and the background data of the examinee as input data.

The OCT image formed by the image forming unit is an example of the “second OCT image” according to the embodiments.

### [Operation Example]

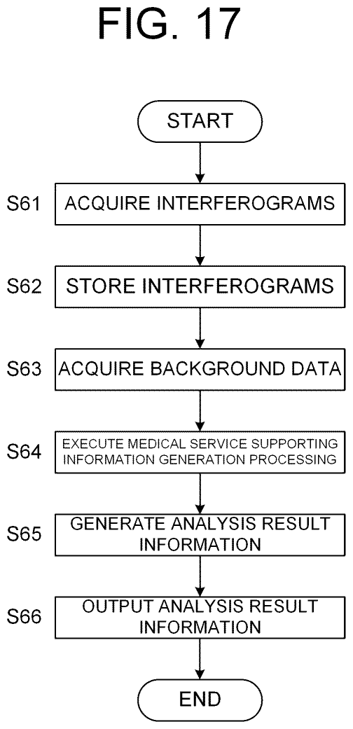

FIG. 17 shows a flowchart of an example of an operation of the ophthalmic information processing apparatus according to the present modification example. The storage unit according to the present modification example or a storage unit (not shown) in the information processor stores a computer program for realizing the processing shown in FIG. 17. One or more processors configured to realize the functions of the ophthalmic information processing apparatus according to the present modification example operates according to the computer program, and thereby the one or more processors perform the processing shown in FIG. 17.

In FIG. 17, it is assumed that the data set of the analysis model is expanded in advance in the working memory of the information processor according to the present modification example, the data set being read out from the storage unit according to the present modification example or the storage unit (not shown) in the information processor according to the present modification example, in the same manner as in FIG. 6.

### (S61: Acquire Interferograms)

First, the main controller according to the present modification example controls the communication unit 130 to establish the communication connection with the communication unit 13 in the OCT apparatus 10, in the same manner as in step S11.

The main controller acquires the one or more interferograms stored in the OCT apparatus 10 via the communication path established with the communication unit 13 in the OCT apparatus 10.

### (S62: Store Interferograms)

Subsequently, the main controller stores the one or more interferograms acquired in step S61 in the storage unit, in the same manner as in step S12.

### (S63: Acquire Background Data)

Sequentially, the main controller acquires the background data of the examinee, and stores the acquired background data in the storage unit. For example, the main controller controls the communication unit 130 to acquire the background data from an external server apparatus in which the background data of the examinee has been stored in advance. Alternatively, the main controller acquires the background data designated by the user using the operating apparatus 180. Alternatively, the main controller acquires the background data from an electronic health record. In some embodiments, the main controller controls the communication unit 130 to acquires the background data from the electronic health record stored in an external server apparatus, and to acquire the acquired background data to which background data designated by the user using the operating apparatus 180 has been added.

### (S64: Execute Medical Service Supporting Information Generation Processing)

Subsequently, the main controller controls the analyzer in the information processor to execute the medical service supporting information generation processing using the analysis model with the one or more interferograms stored in step S62 and the background data acquired in step S63 as input data.

The analyzer outputs the medical service supporting information described above as the output data, by inputting the one or more interferograms and the background data to the data set of the analysis model expanded in advance in the working memory.

In some embodiments, subsequent to step S62, the main controller controls the image forming unit not shown to form the OCT image based on the one or more interferograms stored in step S62. In step S64, the main controller controls the analyzer in the information processor to execute the medical service supporting information generation processing using the analysis model with the one or more interferograms, the formed OCT image(s), and the background data acquired in step S63 as input data.

### (S65: Generate Analysis Result Information)

Next, the main controller controls the analysis result information generator in the information processor to generate the analysis result information of the eye of the examinee using the medical service supporting information obtained in step S64.

The analysis result information generator generates the analysis result information including at least a part of the medical service supporting information, as described above.

### (S66: Output Analysis Result Information)

Next, the main controller outputs the analysis result information generated in step S65, in the same manner as in step S15.

This terminates the processing of the ophthalmic information processing apparatus according to the present modification example (END).

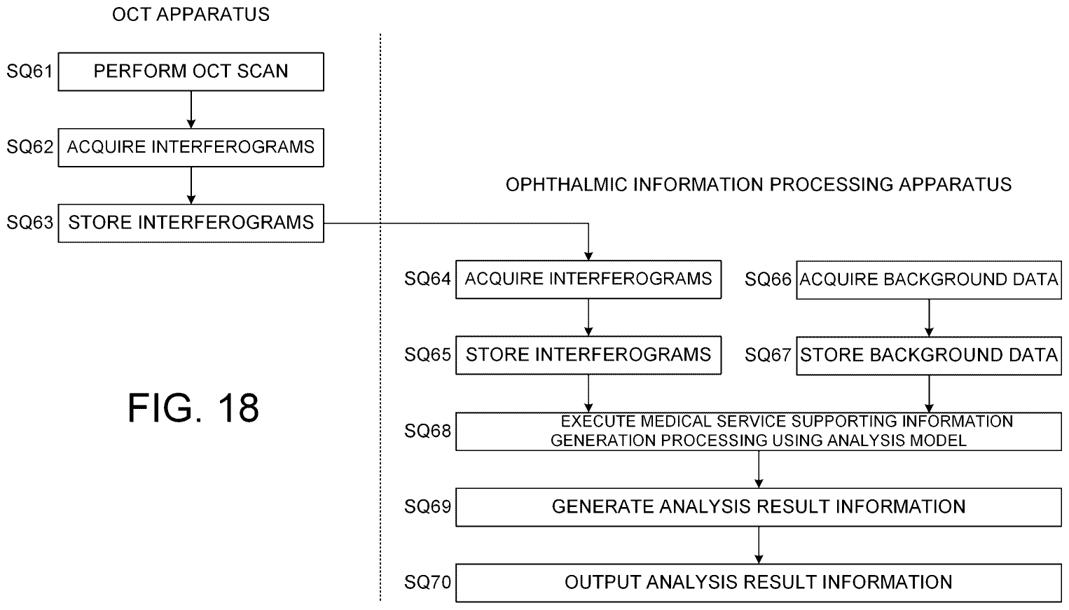

FIG. 18 shows an example of an operation sequence of the ophthalmic system 1 according to the fifth modification example of the embodiments. In FIG. 18, it is assumed that the analysis model has already been built by the analysis model building unit according to the present modification example and the data set of the analysis model has been stored in the storage unit according to the present modification example or the storage unit (not shown) in the information processor according to the present modification example, in the same manner as in FIG. 7.

In the OCT apparatus 10, the controller 11 controls the OCT optical system 12 to perform OCT scan on the eye of the examinee under photographing conditions (photographing light quantity, photographing region) set in advance (SQ61).

The controller 11 acquires the one or more interferograms by sequentially acquiring the detection results of the interference light acquired by the OCT scan (SQ62), and stores the acquired one or more interferograms in the storage unit (not shown) in the controller 11 (SQ63).

In the ophthalmic information processing apparatus according to the present modification example, the controller controls the communication unit to establish the communication connection with the OCT apparatus 10 to acquire the one or more interferograms from the OCT apparatus 10 (SQ64). After the one or more interferograms have been acquired by the OCT apparatus 10, the controller 11 may control the communication unit 13 to establish the communication connection with the ophthalmic information processing apparatus according to the present modification example and to transmit the one or more interferograms to the ophthalmic information processing apparatus.

The controller in the ophthalmic information processing apparatus according to the present modification example stores the one or more interferograms acquired from the OCT apparatus 10 in the storage unit according to the present modification example or the storage unit (not shown) in the information processor according to the present modification example (SQ65).

On the other hand, in the ophthalmic information processing apparatus according to the present modification example, the controller acquires the background data of the examinee, before acquiring the one or more interferograms from the OCT apparatus 10, or after acquiring the one or more interferograms from the OCT apparatus 10 (SQ66). The controller stores the acquired background data in the storage unit (SQ67).

Subsequently, the controller controls the analyzer according to the present modification example to execute the medical service supporting information generation processing, using the one or more interferograms and the background data as input data (SQ68). The data set of the analysis model has been read out from the storage unit or the storage unit (not shown) in the information processor, and has already been expanded in the working memory. The analyzer outputs the medical service supporting information described above as the output data, by inputting the one or more interferograms and the background data to the data set of the analysis model expanded in the working memory.

In some embodiments, the controller controls the image forming unit not shown to form the OCT image based on the one or more interferograms acquired from the OCT apparatus 10. The controller controls the analyzer according to the present modification example to execute the medical service supporting information generation processing, using the one or more interferograms, the OCT image(s), and the background data as input data. The analyzer outputs the medical service supporting information described above as the output data, by inputting the one or more interferograms, the OCT image(s), and the background data to the data set of the analysis model expanded in the working memory.

The controller controls the analysis result information generator to generate the analysis result information using at least a part of the medical service supporting information output from the analysis model (SQ69). After that, the controller controls the display apparatus 190, etc. to output the generated analysis result information (SQ70).

As explained above, according to the present modification example, in the same manner as in the embodiments, the medical service supporting information is generated directly from the one or more interferograms and the background data using the analysis model obtained by machine learning. This eliminates the need for complex signal processing on the acquired interferograms. As a result, the loss of information amount accompanying signal processing can be suppressed, and the analysis result of the eye of the examinee can be obtained with good accuracy and precision, or with high precision and efficiency. Further, since the medical service supporting information is generated using the background data, the analysis result of the examinee can be obtained with higher accuracy and higher reliability. Furthermore, according to the present modification example, the same effects as those described above can be obtained in the same manner as in the embodiments.

It should be noted that in the embodiment described above, and the first modification example to the fourth modification example of the embodiments, the medical service supporting information may be output by inputting input data into the analysis model. Here, the input data is data to which background data has been added to the one or more interferograms, or the one or more interferograms and the OCT image(s).

### Sixth Modification Example

In the embodiments or the modification examples thereof, a case in which the ophthalmic system includes the OCT apparatus and the ophthalmic information processing apparatus has been mainly described. However, the configuration of the ophthalmic system according to the embodiments is not limited to this. For example, the ophthalmic system according to the embodiments may have the configuration of the ophthalmic apparatus in which the functions of the OCT apparatus and the functions of the ophthalmic information processing apparatus are housed in the same housing.

Hereinafter, the sixth modification example of the embodiments will be described focusing on the differences from the embodiments.

The ophthalmic apparatus according to the sixth modification example of the embodiments is an example of the ophthalmic system having the functions of the OCT apparatus 10 according to the embodiments and the functions of the ophthalmic information processing apparatus according to any one of the first to fifth modification examples of the embodiments.

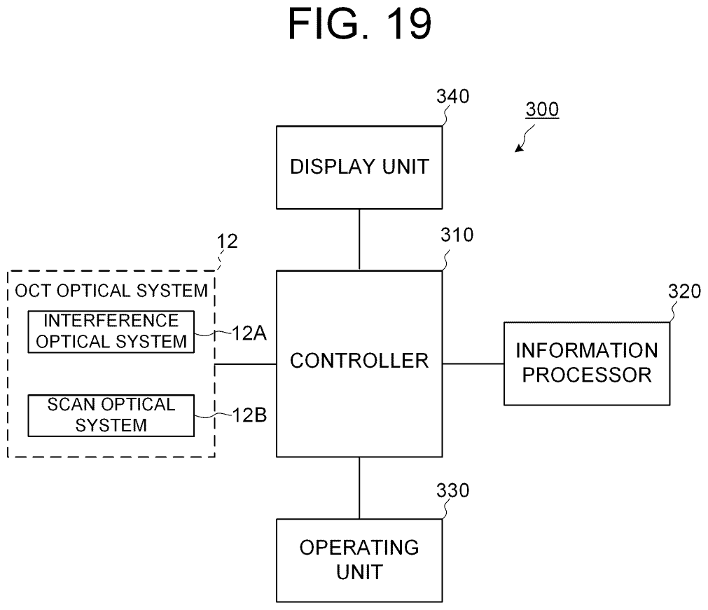

FIG. 19 shows a block diagram of an example of a configuration of an ophthalmic apparatus 300 according to the sixth modification example of the embodiments. In FIG. 19, like reference numerals designate like parts as in FIG. 2, and the redundant explanation may be omitted as appropriate.

The ophthalmic apparatus 300 according to the present modification example includes the OCT optical system 12, a controller 310, and an information processor 320, an operating unit 330, and a display unit 340. In the ophthalmic apparatus 300, at least one of the operating unit 330 or the display unit 340 may be omitted.

The controller 310 has the function of controlling the OCT optical system 12 among the functions of the controller 11 in FIG. 2, and the function of controlling the information processor 120 among the functions of the controller 110 in FIG. 3. The information processor 320 has the same functions as the information processor in any of the embodiments and the first to the fifth modification examples of the embodiments. The operating unit 330 has the same functions as the operating apparatus 180 in FIG. 1. The display unit 340 has the same function as the display apparatus 190 in FIG. 1.

The display unit 340 is an example of the “display means” according to the embodiments.

According to the present modification example, the medical service supporting information is generated directly from at least the one or more interferograms using the analysis model obtained by machine learning, in a compact configuration. This eliminates the need for complex signal processing on the acquired interferograms. As a result, the loss of information amount accompanying signal processing can be suppressed, and the analysis result of the eye of the examinee can be obtained with good accuracy and precision, or with high precision and efficiency. In addition, according to the present modification example, the same effects as those described above can be obtained in the same manner as in the embodiments.

### [Actions]

The ophthalmic information processing apparatus, the ophthalmic system, the ophthalmic information processing method, and the program according to the embodiments will be described.

The first aspect of the embodiments is an ophthalmic information processing apparatus (100, 100a) including an acquisition unit (communication unit 130) and an information processor (120, 120a, 320). The acquisition unit is configured to acquire one or more interferograms obtained by performing OCT scan on an eye of an examinee. The information processor is configured to execute generation processing of medical service supporting information that supports a provision of a medical service for the examinee, based on the one or more interferograms. The information processor is configured to execute at least a part of the generation processing using a learned model (analysis model) generated in advance by performing machine learning.

According to such a configuration, the medical service supporting information is generated directly from the one or more interferograms acquired by performing OCT scan on the eye of the examinee, using the learned model obtained by machine learning. Thereby, the medical service supporting information can be acquired with high speed and accuracy, without performing complex signal processing on the acquired interferograms. As a result, the information that supports the provision of medical services to examinees can be acquired with good accuracy and precision, or can be acquired efficiently with high accuracy. Further, since the medical service supporting information can be systematically acquired from the interferograms, the medical service supporting information can be acquired systematically even when the models of OCT devices for acquiring the interferograms differ.

In the second aspect of the embodiments, in the first aspect, the medical service supporting information includes at least one of a first OCT image of the eye or an analysis result of a morphology of a tomographic structure of the eye.

According to such a configuration, at least one of the OCT image or the analysis result of the morphology of the tomographic structure of the eye of the examinee can be acquired with good accuracy and precision, or can be acquired efficiently with high accuracy. Further, at least one of the OCT image or the analysis result of the morphology of the tomographic structure of the eye of the examinee can be systematically acquired from the interferograms. Thereby, at least one of the OCT image or the analysis result of the morphology of the tomographic structure of the eye of the examinee can be systematically acquired, even when the models of OCT devices for acquiring the interferograms differ.

In the third aspect of the embodiments, in the first aspect or the second aspect, the analysis result includes supporting information that supports a determination of presence or absence of a disease, supporting information that supports a determination of presence of absence of a risk of developing a disease, supporting information that supports a determination of a type of a disease, supporting information that supports determination of necessity of an examination, or supporting information that supports a decision of a treatment of a disease.

According to such a configuration, the supporting information that supports the determination of presence or absence of the disease, the determination of presence of absence of the risk of developing the disease, the determination of the type of the disease, the determination of necessity of the examination, or the decision of the treatment of the disease can be acquired with good accuracy and precision, or can be acquired efficiently with high accuracy. Further, the supporting information described above can be systematically acquired from the interferograms. Thereby, the supporting information described above can be systematically acquired, even when the models of OCT devices for acquiring the interferograms differ.

In the fourth aspect of the embodiments, in any one of the first aspect to the third aspect, the learned model is a learned model generated in advance so as to output medical service supporting information by performing machine learning using at least interferograms as input.

According to such a configuration, the medical service supporting information can be acquired directly from the one or more interferograms with high speed and accuracy in a compact configuration.

The fifth aspect of the embodiments, in any one of the first aspect to the fourth aspect, further includes an image forming unit (140) and an analysis result information generator (220). The image forming unit is configured to form a second OCT image of the eye by performing at least Fourier transformation based on the one or more interferograms. The analysis result information generator is configured to generate analysis result information based on the second OCT image and the medical service supporting information generated by the information processor.

According to such a configuration, while forming the second OCT image of the eye by performing at least Fourier transformation based on the one or more interferograms that are input to the learned model, the analysis result information is generated using the formed second OCT image. Thereby, the analysis result of the morphology of the tomographic structure of the eye of the examinee can be acquired with good accuracy and precision, or can be acquired efficiently with high accuracy. Further, the analysis result of the morphology of the tomographic structure of the eye of the examinee can be systematically acquired from the interferograms. Thereby, the analysis result of the morphology of the tomographic structure of the eye of the examinee can be systematically acquired, even when the models of OCT devices for acquiring the interferograms differ.

In the sixth aspect of the embodiments, in any one of the first aspect to the fourth aspect, the information processor is configured to execute generation processing of the medical service supporting information based on the one or more interferograms and background data of the examinee. The learned model is a learned model generated in advance so as to output medical service supporting information by performing machine learning using one or more interferograms and background data as input.

According to such a configuration, the medical service supporting information is generated directly from the one or more interferograms, using the learned model obtained by machine learning using the background data of the examinee. Thereby, the medical service supporting information can be acquired with higher accuracy. As a result, the information that supports the provision of medical services to examinees can be acquired with good accuracy and precision, or can be acquired efficiently with higher accuracy.

The seventh aspect of the embodiments, in any one of the first aspect to the fourth aspect, further includes an image forming unit (140). The image forming unit is configured to form a second OCT image of the eye by performing at least Fourier transformation based on the one or more interferograms. The information processor is configured to execute generation processing of the medical service supporting information based on the one or more interferograms and the second OCT image (OCT image formed by the image forming unit). The learned model is a learned model generated in advance so as to output medical service supporting information by performing machine learning using one or more interferograms and second OCT images as input.

According to such a configuration, the medical service supporting information is generated directly from the one or more interferograms, using the learned model obtained by machine learning using the second OCT image formed by performing at least Fourier transformation. Thereby, the medical service supporting information can be acquired with good accuracy and precision, or can be acquired efficiently with higher accuracy. Further, the medical service supporting information can be systematically acquired from the interferograms. Thereby, the medical service supporting information can be systematically acquired, even when the models of OCT devices for acquiring the interferograms differ.

In the eighth aspect of the embodiments, in the seventh aspect, the information processor is configured to execute generation processing of the medical service supporting information based on the one or more interferograms, the second OCT image, and background data of the examinee. The learned model is a learned model generated in advance so as to output medical service supporting information by performing machine learning using one or more interferograms, second OCT images, and background data as input. For example, the second OCT image is formed by the image forming unit, by performing at least Fourier transformation based on the one or more interferograms that are input to the learned model.

According to such a configuration, the medical service supporting information is generated directly from the one or more interferograms, using the learned model obtained by machine learning using the OCT image and the background data of the examinee. Thereby, the medical service supporting information can be acquired with higher accuracy. As a result, the information that supports the provision of medical services to examinees can be acquired with good accuracy and precision, or can be acquired efficiently with higher accuracy.

The ninth aspect of the embodiments, in the seventh aspect or the eighth aspect, further includes a display controller (controller 110, 110a, 310, main controller 111, 111a) configured to display the second OCT image generated by the image forming unit on a display means (display apparatus 190, display unit 340).

According to such a configuration, the medical service supporting information is generated using the learned model with at least the one or more interferograms, and is displayed the OCT image formed based on the one or more interferograms on the display means. Thereby, the medical service supporting information can be acquired with higher accuracy while acquiring the OCT image in the same manner as the conventional method. As a result, the information that supports the provision of medical services to examinees can be acquired with good accuracy and precision, or can be acquired efficiently with higher accuracy.

The tenth aspect of the embodiments is an ophthalmic system (1, ophthalmic apparatus 300) including an OCT optical system (12) and the ophthalmic information processing apparatus in any one of the first aspect to the ninth aspect. The OCT optical system is configured to perform OCT scan on an eye of an examinee. The ophthalmic information processing apparatus is configured to acquire the one or more interferograms from the OCT optical system.

According to such a configuration, the ophthalmic system that can acquire the medical service supporting information with high speed and accuracy in a compact configuration can be provided. As a result, the information that supports the provision of medical services to examinees can be acquired with good accuracy and precision, or can be acquired efficiently with high accuracy. Further, since the medical service supporting information can be systematically acquired from the interferograms, the medical service supporting information can be acquired systematically even when the models of OCT devices for acquiring the interferograms differ.

The eleventh aspect of the embodiments is an ophthalmic information processing method includes an acquisition step and an information processing step. The acquisition step is performed to acquire one or more interferograms obtained by performing OCT scan on an eye of an examinee. The information processing step is performed to execute generation processing of medical service supporting information that supports a provision of a medical service for the examinee, based on the one or more interferograms. The information processing step is performed to execute at least a part of the generation processing described above using a learned model generated in advance by performing machine learning.

According to such a method, the medical service supporting information is generated directly from the one or more interferograms acquired by performing OCT scan on the eye of the examinee, using the learned model obtained by machine learning. Thereby, the medical service supporting information can be acquired with high speed and accuracy, without performing complex signal processing on the acquired interferograms. As a result, the information that supports the provision of medical services to examinees can be acquired with good accuracy and precision, or can be acquired efficiently with high accuracy. Further, since the medical service supporting information can be systematically acquired from the interferograms, the medical service supporting information can be acquired systematically even when the models of OCT devices for acquiring the interferograms differ.

In the twelfth aspect of the embodiments, in the eleventh aspect, the medical service supporting information includes at least one of a first OCT image of the eye or an analysis result of a morphology of a tomographic structure of the eye.

According to such a method, at least one of the OCT image or the analysis result of the morphology of the tomographic structure of the eye of the examinee can be acquired with good accuracy and precision, or can be acquired efficiently with high accuracy. Further, at least one of the OCT image or the analysis result of the morphology of the tomographic structure of the eye of the examinee can be systematically acquired from the interferograms. Thereby, at least one of the OCT image or the analysis result of the morphology of the tomographic structure of the eye of the examinee can be systematically acquired, even when the models of OCT devices for acquiring the interferograms differ.

In the thirteenth aspect of the embodiments, in the eleventh aspect or the twelfth aspect, the analysis result includes supporting information that supports a determination of presence or absence of a disease, supporting information that supports a determination of presence of absence of a risk of developing a disease, supporting information that supports a determination of a type of a disease, supporting information that supports determination of necessity of an examination, or supporting information that supports a decision of a treatment of a disease.

According to such a method, the supporting information that supports the determination of presence or absence of the disease, the determination of presence of absence of the risk of developing the disease, the determination of the type of the disease, the determination of necessity of the examination, or the decision of the treatment of the disease can be acquired with good accuracy and precision, or can be acquired efficiently with high accuracy. Further, the supporting information described above can be systematically acquired from the interferograms. Thereby, the supporting information described above can be systematically acquired, even when the models of OCT devices for acquiring the interferograms differ.

In the fourteenth aspect of the embodiments, in any one of the eleventh aspect to the thirteenth aspect, the learned model is a learned model generated in advance so as to output medical service supporting information by performing machine learning using at least interferograms as input.

According to such a method, the medical service supporting information can be acquired directly from the one or more interferograms with high speed and accuracy with a simple method.

The fifteenth aspect of the embodiments, in any one of the eleventh aspect to the fourteenth aspect, further includes an image forming step and an analysis result information generation step. The image forming step is performed to form a second OCT image of the eye by performing at least Fourier transformation based on the one or more interferograms. The analysis result information generating step is performed to generate analysis result information based on the second OCT image and the medical service supporting information generated in the information processing step.

According to such a method, while forming the second OCT image of the eye by performing at least Fourier transformation based on the one or more interferograms that are input to the learned model, the analysis result information is generated using the formed second OCT image. Thereby, the analysis result of the morphology of the tomographic structure of the eye of the examinee can be acquired with good accuracy and precision, or can be acquired efficiently with high accuracy. Further, the analysis result of the morphology of the tomographic structure of the eye of the examinee can be systematically acquired from the interferograms. Thereby, the analysis result of the morphology of the tomographic structure of the eye of the examinee can be systematically acquired, even when the models of OCT devices for acquiring the interferograms differ.

In the sixteenth aspect of the embodiments, in any one of the eleventh aspect to the fourteenth aspect, the information processing step is performed to execute generation processing of the medical service supporting information based on the one or more interferograms and background data of the examinee. The learned model is a learned model generated in advance so as to output medical service supporting information by performing machine learning using one or more interferograms and background data as input.

According to such a method, the medical service supporting information is generated directly from the one or more interferograms, using the learned model obtained by machine learning using the background data of the examinee. Thereby, the medical service supporting information can be acquired with higher accuracy. As a result, the information that supports the provision of medical services to examinees can be acquired with good accuracy and precision, or can be acquired efficiently with higher accuracy.

The seventeenth aspect of the embodiments, in any one of the eleventh aspect to the fourteenth aspect, further includes an image forming step. The image forming step is performed to form a second OCT image of the eye by performing at least Fourier transformation based on the one or more interferograms. The information processing step is performed to execute generation processing of the medical service supporting information based on the one or more interferograms and the second OCT image. The learned model is a learned model generated in advance so as to output medical service supporting information by performing machine learning using one or more interferograms and second OCT images as input.

According to such a method, the medical service supporting information is generated directly from the one or more interferograms, using the learned model obtained by machine learning using the second OCT image formed by performing at least Fourier transformation. Thereby, the medical service supporting information can be acquired with good accuracy and precision, or can be acquired efficiently with higher accuracy. Further, the medical service supporting information can be systematically acquired from the interferograms. Thereby, the medical service supporting information can be systematically acquired, even when the models of OCT devices for acquiring the interferograms differ.

In the eighteenth aspect of the embodiments, in the seventeenth aspect, the information processing step is performed to execute generation processing of the medical service supporting information based on the one or more interferograms, the second OCT image, and background data of the examinee. The learned model is a learned model generated in advance so as to output medical service supporting information by performing machine learning using one or more interferograms, second OCT images, and background data as input.

According to such a method, the medical service supporting information is generated directly from the one or more interferograms, using the learned model obtained by machine learning using the OCT image and the background data of the examinee. Thereby, the medical service supporting information can be acquired with higher accuracy. As a result, the information that supports the provision of medical services to examinees can be acquired with good accuracy and precision, or can be acquired efficiently with higher accuracy.

The nineteenth aspect of the embodiments, in the seventeenth aspect or the eighteenth aspect, further includes a display control step of displaying the second OCT image formed in the image forming step on a display means (display apparatus 190, display unit 340).

According to such a method, the medical service supporting information is generated using the learned model with at least the one or more interferograms, and is displayed the OCT image formed based on the one or more interferograms on the display means. Thereby, the medical service supporting information can be acquired with higher accuracy while acquiring the OCT image in the same manner as the conventional method. As a result, the information that supports the provision of medical services to examinees can be acquired with good accuracy and precision, or can be acquired efficiently with higher accuracy.

The twentieth aspect of the embodiments is a program of causing a computer to execute each step of the ophthalmic information processing method of any one of the eleventh aspect to the nineteenth aspect.

According to such a program, the medical service supporting information is generated directly from the one or more interferograms acquired by performing OCT scan on the eye of the examinee, using the learned model obtained by machine learning. Thereby, the medical service supporting information can be acquired with high speed and accuracy, without performing complex signal processing on the acquired interferograms. As a result, the information that supports the provision of medical services to examinees can be acquired with good accuracy and precision, or can be acquired efficiently with high accuracy. Further, since the medical service supporting information can be systematically acquired from the interferograms, the medical service supporting information can be acquired systematically even when the models of OCT devices for acquiring the interferograms differ.

### <Others>

The embodiment described above is merely an example for implementing the present invention. Those who intend to implement the present invention can apply any modification, omission, addition, or the like within the scope of the gist of the present invention.

In some embodiments, a program for causing a computer (processor) to execute the ophthalmic information processing method described above is provided. Such a program can be stored in any non-transitory recording medium (storage medium) that can be read by a computer. Examples of the recording medium include a semiconductor memory, an optical disk, a magneto-optical disk (CD-ROM, DVD-RAM, DVD-ROM, MO, etc.), a magnetic storage medium (hard disk, floppy (registered trade mark) disk, ZIP, etc.), and the like. Further, the program may be transmitted and received through a network such as the Internet, LAN, etc.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ophthalmic information processing apparatus, comprising:
an acquisition unit configured to acquire one or more interferograms obtained by performing OCT scan on an eye of an examinee; and
an information processor configured to execute generation processing of medical service supporting information that supports a provision of a medical service for the examinee, based on the one or more interferograms, wherein
the information processor is configured to execute at least a part of the generation processing using a learned model generated in advance by performing machine learning.

2. The ophthalmic information processing apparatus of claim 1, wherein
the medical service supporting information includes at least one of a first OCT image of the eye or an analysis result of a morphology of a tomographic structure of the eye.

3. The ophthalmic information processing apparatus of claim 2, wherein
the analysis result includes supporting information that supports a determination of presence or absence of a disease, supporting information that supports a determination of presence of absence of a risk of developing a disease, supporting information that supports a determination of a type of a disease, supporting information that supports determination of necessity of an examination, or supporting information that supports a decision of a treatment of a disease.

4. The ophthalmic information processing apparatus of claim 1, wherein
the learned model is a learned model generated in advance so as to output medical service supporting information by performing machine learning using at least interferograms as input.

5. The ophthalmic information processing apparatus of claim 1, further comprising:
an image forming unit configured to form a second OCT image of the eye by performing at least Fourier transformation based on the one or more interferograms; and
an analysis result information generator configured to generate analysis result information based on the second OCT image and the medical service supporting information generated by the information processor.

6. The ophthalmic information processing apparatus of claim 1, wherein
the information processor is configured to execute generation processing of the medical service supporting information based on the one or more interferograms and background data of the examinee, and
the learned model is a learned model generated in advance so as to output medical service supporting information by performing machine learning using one or more interferograms and background data as input.

7. The ophthalmic information processing apparatus of claim 1, further comprising
an image forming unit configured to form a second OCT image of the eye by performing at least Fourier transformation based on the one or more interferograms, wherein
the information processor is configured to execute generation processing of the medical service supporting information based on the one or more interferograms and the second OCT image, and
the learned model is a learned model generated in advance so as to output medical service supporting information by performing machine learning using one or more interferograms and second OCT images as input.

8. The ophthalmic information processing apparatus of claim 7, wherein
the information processor is configured to execute generation processing of the medical service supporting information based on the one or more interferograms, the second OCT image, and background data of the examinee, and
the learned model is a learned model generated in advance so as to output medical service supporting information by performing machine learning using one or more interferograms, second OCT images, and background data as input.

9. The ophthalmic information processing apparatus of claim 7, further comprising
a display controller configured to display the second OCT image generated by the image forming unit on a display means.

10. An ophthalmic system, comprising:
an OCT optical system configured to perform OCT scan on an eye of an examinee; and
an ophthalmic information processing apparatus configured to acquire one or more interferograms from the OCT optical system, wherein
the ophthalmic information processing apparatus comprises:
an acquisition unit configured to acquire one or more interferograms obtained by performing OCT scan on the eye of the examinee; and
an information processor configured to execute generation processing of medical service supporting information that supports a provision of a medical service for the examinee, based on the one or more interferograms, wherein
the information processor is configured to execute at least a part of the generation processing using a learned model generated in advance by performing machine learning.

11. An ophthalmic information processing method, comprising:
an acquisition step of acquiring one or more interferograms obtained by performing OCT scan on an eye of an examinee; and
an information processing step of executing generation processing of medical service supporting information that supports a provision of a medical service for the examinee, based on the one or more interferograms, wherein
the information processing step is performed to execute at least a part of the generation processing using a learned model generated in advance by performing machine learning.

12. The ophthalmic information processing method of claim 11, wherein
the medical service supporting information includes at least one of a first OCT image of the eye or an analysis result of a morphology of a tomographic structure of the eye.

13. The ophthalmic information processing method of claim 12, wherein
the analysis result includes supporting information that supports a determination of presence or absence of a disease, supporting information that supports a determination of presence of absence of a risk of developing a disease, supporting information that supports a determination of a type of a disease, supporting information that supports determination of necessity of an examination, or supporting information that supports a decision of a treatment of a disease.

14. The ophthalmic information processing method of claim 11, wherein
the learned model is a learned model generated in advance so as to output medical service supporting information by performing machine learning using at least interferograms as input.

15. The ophthalmic information processing method of claim 11, further comprising:
an image forming step of forming a second OCT image of the eye by performing at least Fourier transformation based on the one or more interferograms; and
an analysis result information generating step of generating analysis result information based on the second OCT image and the medical service supporting information generated in the information processing step.

16. The ophthalmic information processing method of claim 11, wherein
the information processing step is performed to execute generation processing of the medical service supporting information based on the one or more interferograms and background data of the examinee, and
the learned model is a learned model generated in advance so as to output medical service supporting information by performing machine learning using one or more interferograms and background data as input.

17. The ophthalmic information processing method of claim 11, further comprising
an image forming step of forming a second OCT image of the eye by performing at least Fourier transformation based on the one or more interferograms, wherein
the information processing step is performed to execute generation processing of the medical service supporting information based on the one or more interferograms and the second OCT image, and
the learned model is a learned model generated in advance so as to output medical service supporting information by performing machine learning using one or more interferograms and second OCT images as input.

18. The ophthalmic information processing method of claim 17, wherein
the information processing step is performed to execute generation processing of the medical service supporting information based on the one or more interferograms, the second OCT image, and background data of the examinee, and
the learned model is a learned model generated in advance so as to output medical service supporting information by performing machine learning using one or more interferograms, second OCT images, and background data as input.

19. The ophthalmic information processing method of claim 17, further comprising
a display control step of displaying the second OCT image formed in the image forming step on a display means.

20. A computer readable non-transitory recording medium in which a program for causing a computer to execute each step of an ophthalmic information processing method is recorded, wherein
the ophthalmic information processing method comprising:
an acquisition step of acquiring one or more interferograms obtained by performing OCT scan on an eye of an examinee; and
an information processing step of executing generation processing of medical service supporting information that supports a provision of a medical service for the examinee, based on the one or more interferograms, wherein
the information processing step is performed to execute at least a part of the generation processing using a learned model generated in advance by performing machine learning.
